(19) United States
(12) Patent Application Publication
(10) Pub. No.: US 2018/0240552 A1
(43) Pub. Date: Aug. 23, 2018

(54) SYSTEM AND METHOD FOR MANAGING TREATMENT PLANS

(71) Applicant: Penexa, LLC, Pleasant Hill, CA (US)

(72) Inventors: Robert van Tuyl, Pleasanton, CA (US); Nhan T. Nguyen, Lafayette, CA (US)

(21) Appl. No.: 15/900,770

(22) Filed: Feb. 20, 2018

Related U.S. Application Data
(60) Provisional application No. 62/461,184, filed on Feb. 20, 2017.

(51) Int. Cl. G16H 50/20; G06N 5/02

(52) U.S. Cl. CPC G16H 50/20; G06N 5/02

(57) ABSTRACT

Disclosed is an improved approach to implement continuous home and community care processes in the healthcare industry. Embodiments of the present disclosure describe systems and processes for standardization of treatments and outcomes using a clinical intelligence engine. An embodiment of this disclosure is a healthcare technology platform that enables the distribution of decision making from a central location to the edge of the system, thereby improving the overall treatment efficacy.

### RELATED APPLICATIONS

This present application claims the benefit of U.S. Provisional Application No. 62/461,184 filed Feb. 20, 2017, titled “SYSTEM AND METHOD FOR MANAGING TREATMENT PLANS”, which is hereby incorporated by reference in its entirety.

### FIELD

This disclosure concerns methods, computer program products, and computer systems for supporting continuous home and community care in the healthcare industry by managing treatment plans.

### BACKGROUND

Patients with health issues may benefit from ongoing therapy programs that are directed to reducing deficits and issues associated to the health issues. Certain health issues may require periodic therapy sessions with practitioner(s) over a period of time with the need for periodic assessments, reassessments and adjustments based on an individual's progress throughout the periodic therapy sessions.

Most legacy solutions consist of manual implementations of treatment plans/therapy programs that involve paper data collection forms for capturing results, where the practitioner uses a copy of a template form that represents a treatment plan that was selected by a program supervisor from a set of pre-defined treatment plans from, as an example, a physical workbook based on a client's (e.g., patient's) diagnosis and assessments.

A key shortcoming of the legacy process is that paper-based treatment plans and manual data entry onto paper-based treatment plans for capturing results can be cumbersome, error prone, and generally not very efficient because, for the most part, data received from the treatment and/or analysis of the client are stored on physical paper via manual data collection techniques (e.g., written on paper), making it difficult to learn or improve on existing process based on results obtained from implementation of the treatment plans over a large number of clients. Moreover, the creation and adjustment of the treatment plans are not flexible because the treatment plans are based on a process of copying pre-defined paper templates (with inherent limited granularity). Clinical managers are limited to selecting treatment plans from a set of pre-defined templates that are infrequently updated. Practitioners often copy these pre-defined templates from published workbooks, and fill out these paper copies during client evaluation sessions.

The legacy process does not support client specific customization and periodic/frequent adjustments that may improve the overall treatment efficacy in a shorter amount of time. Legacy treatment processes include several manual steps as well as paper data entry at several points during the treatment process. The lack of automation and data integration in the legacy process significantly limits the efficiency of the end-to-end process. Since there are no interoperable standards in legacy processes, automation of the processes and data mining of the results to improve the overall efficacy of the treatment program as a whole is greatly limited.

Furthermore, legacy processes are also limited to treatment performed by a client only in the presence of a practitioner. For example, with the growing number of Autism Spectrum Disorder (ASD) cases, the one on one treatment performed by a practitioner only in the presence of a client may result in less frequent touch points between the practitioner(s) with a specific client because the number of new ASD cases may quickly surpass the number of practitioners available for the one on one treatments, therefore limiting the amount of one on one treatments provided because of the limited number of practitioners. Hence the continuous care may be less frequent. Another problem that is also prevalent is that parents are generally not involved with the treatment execution because in the legacy process, the practitioner provides the treatment execution with the client—not the parent(s).

Another drawback of legacy systems is the lack of end-to-end data integration that further limits the overall visibility of treatment results to viewable by clinical managers and healthcare providers, which further limits the efficacy of the overall treatment. Additionally, paper-based processes are also hard to secure which can make HIPAA compliance very challenging. Moreover, in the case of ASD, treatments tend to be expensive, and inefficient.

Therefore, there is a need for an improved approach to implement a technology-based infrastructure that addresses the above-described problems.

### SUMMARY

Embodiments of the present disclosure provide an improved approach to implement continuous home and community care processes in the healthcare industry. Embodiments of the present disclosure describe systems and processes for standardization of treatments and outcomes using a clinical intelligence engine. Additionally, embodiments of the present disclosure may leverage Artificial Intelligence (AI) machine learning capabilities to enhance and expand a clinical knowledge base in using data/information feedback from clinical applications of treatment plans and industry published researches. Another embodiment of the present disclosure describes real time distribution of client treatment information from a central location to mobile applications independent of care location. An embodiment of this disclosure is a healthcare technology platform that enables the distribution of decision making from a central location to the edge of the system, thereby improving the overall treatment efficacy.

In one embodiment, a system, computer program product, and method may include receiving patient assessment information, generating a candidate treatment plan by applying the patient assessment information against clinical rules selected from a clinical knowledge base, approving a treatment plan from the candidate treatment plan via a workflow engine, receiving results data from implementation of the approved treatment plan with a patient via a mobile device, generating clinical rule recommendations, approving one or more clinical rules from the generated clinical rule recommendations by a clinical standards review team, and updating the clinical knowledge base with the one or more clinical rules approved.

In one or more embodiments, the assessment information comprises a plurality of assessment scores. The candidate treatment plan corresponds to clinical rules retrieved from the clinical knowledge base by a decision engine comprising an expert system. The clinical knowledge base may include clinical rules approved and validated by a clinical review team of practitioners reviewing and validating clinical rules generated by an artificial intelligence system analyzing results data captured by one or more devices.

In one or more embodiments, approving the treatment plan comprises analyzing the candidate treatment plan by a clinical manager using a workflow engine, the workflow engine verifying the treatment plan based at least in part on the assessment information received, and assigning and scheduling one or more treatment plan tasks with one or more behavioral interventionists and the patient. The results received from the implementation of the treatment plan are received into a results database, the results comprising data received from one or more devices collecting patient information. The one or more devices may include a data collection application (DCA) capturing patient information as a result of the implementation of the treatment plan and/or one or more sensors continuously collecting patient information during the implementation of the treatment plan and/or during a course of normal patient activity.

In one or more embodiments, the clinical rule recommendations are generated by an outcome analysis engine based at least in part on the results received from implementation of the treatment plan performed on a plurality of patients, the outcome analysis engine comprising a machine learning system analyzing at least the results received from the implementation of the treatment plan and external data sources comprising structured and unstructured data. The machine learning system implements a deep learning algorithm for generating the clinical rule recommendations. Approving the one or more clinical rules comprises the clinical review team validating the clinical rules via one or more clinical trials.

Each of the individual embodiments described and illustrated herein has discrete components and features that may be readily separated from or combined with the components and features of any of the other several embodiments.

Further details of objects and advantages of the disclosure are described below in the detailed description, drawings, and claims. Both the foregoing general description and the following detailed description are exemplary and explanatory, and are not intended to be limiting as to the scope of the disclosure.

### BRIEF DESCRIPTION OF THE DRAWINGS

In order that the present disclosure is better understood, some embodiments of the disclosure will now be described, by way of example only, with reference to the accompanying drawings.

FIGS. 1A-1B illustrate example architectures and systems for implementing a treatment plan management application, according to some embodiments of the disclosure.

FIG. 2 illustrates a treatment process, according to some embodiments of the disclosure.

FIGS. 3A-3B illustrate example processes for managing treatment plans, according to some embodiments of the disclosure.

FIG. 4 presents a diagram of an end-to-end interaction process, according to some embodiments of the disclosure.

FIG. 5 is a block diagram of an ecosystem of the present disclosure, according to some embodiments of the disclosure.

FIG. 6 is a flow chart of a treatment plan creation process detailing the status of the treatment plan, according to some embodiments of the disclosure.

FIGS. 7A-7B depicts example of DCA graphical user interfaces, according to some embodiments of the disclosure.

FIG. 8 is an example flow chart of a practitioner process of using a DCA, according to some embodiments of the disclosure.

FIG. 9 depicts a block diagram of a Treatment Data Model, according to some embodiments of the disclosure.

FIG. 10 is a block diagram of a Treatment Process Interactions, according to some embodiments of the disclosure.

FIG. 11 depicts an example diagram of different machine learning algorithms suitable for implementing an embodiment of the present disclosure.

FIG. 12 illustrates a block diagram of a computing system suitable for implementing an embodiment of the present disclosure.

### DETAILED DESCRIPTION

Various embodiments are described herein with reference to the figures. It should be noted that the figures are not necessarily drawn to scale and that elements of similar structures or functions are sometimes represented by like reference characters throughout the figures. It should also be noted that the figures are only intended to facilitate the description of the disclosed embodiments—they are not representative of an exhaustive treatment of all possible embodiments, and they are not intended to impute any limitation as to the scope of the claims. In addition, an illustrated embodiment need not portray all aspects or advantages of usage in any particular environment. An aspect or an advantage described in conjunction with a particular embodiment is not necessarily limited to that embodiment and can be practiced in any other embodiments even if not so illustrated. Also, references throughout this specification to “some embodiments” or “other embodiments” refers to a particular feature, structure, material or characteristic described in connection with the embodiments as being included in at least one embodiment. Thus, the appearance of the phrases “in some embodiments” or “in other embodiments” in various places throughout this specification are not necessarily referring to the same embodiment or embodiments.

The embodiments and examples herein describe an extendible system for proposing, approving and managing treatment plans for clients independent of their location via a connected platform that supports a feedback mechanism that enables continuous visibility and treatment plan adjustments by clinical staff. Specifically, the embodiments disclose systems and methods for distributing treatment plans to mobile applications utilized by practitioners and parents to administer treatment plan exercises/tasks and record the results, the results being reported back into the system. Additionally, embodiments and examples herein also describe a system and method using an machine learning engine to analyze results received from a plurality of clients and generating new and/or improved clinical rule recommendations to be validated and approved for future use.

An embodiment of the present disclosure may be directed to a treatment plan management application and system for use in Applied Behavior Analysis (ABA) for patients (hereinafter may be referred to “clients”) diagnosed with Autism Spectrum Disorder (ASD). For simplicity of explanation, the systems and methods of this disclosure will be explained with reference to a treatment management application and system for clients with ASD. One of ordinary skill in the art may appreciate the systems and methods disclosed herein may be applicable to other embodiments which may require a treatment plan management application and system to manage treatment plans implemented on clients over a period of time (e.g., treatment plans having therapy sessions occurring over a time period ranging from months to years).

Behavior analysis focuses on the principles that explain how learning takes place. Positive reinforcement is one such principle. When a behavior is followed by some sort of reward, the behavior is more likely to be repeated. The field of behavior analysis has developed many techniques for increasing useful behaviors and reducing those that may cause harm or interfere with learning.

Applied behavior analysis (ABA) is the use of these techniques and principles to bring about meaningful and positive change in behavior. A wide variety of ABA techniques have been developed for building useful skills in learners with autism—from toddlers through adulthood. These techniques may be used in structured situations such as during a therapy session as well as in “everyday” situations such as family dinnertime or the neighborhood playground. Some ABA therapy sessions involve one-on-one interaction between a practitioner and/or behavioral interventionist (hereinafter may be referred to as a “BI”) and the client.

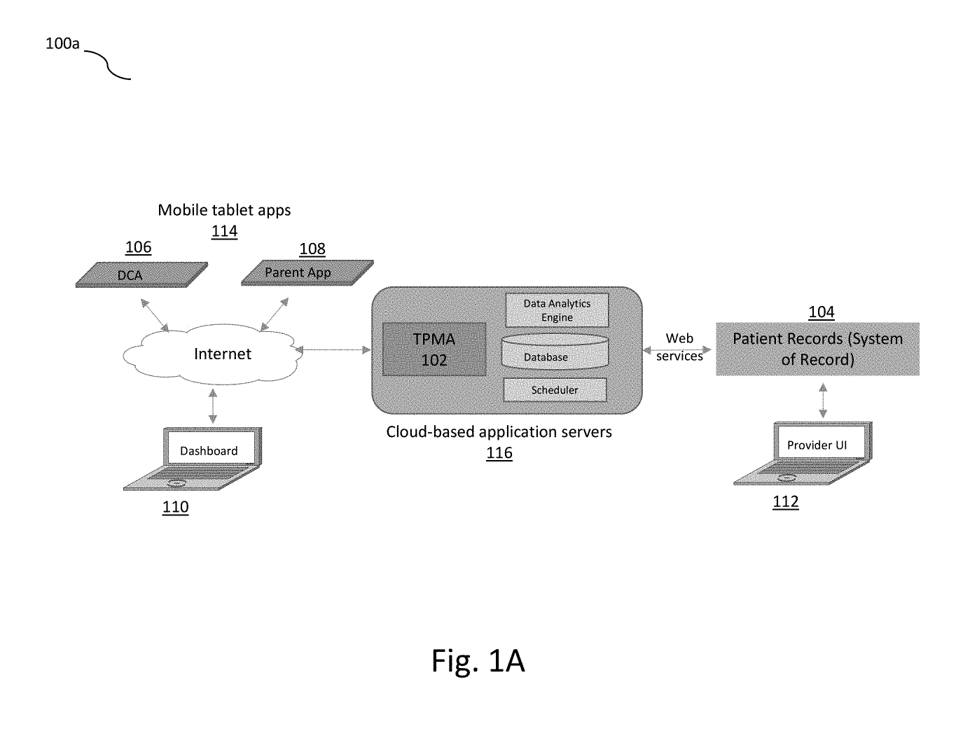

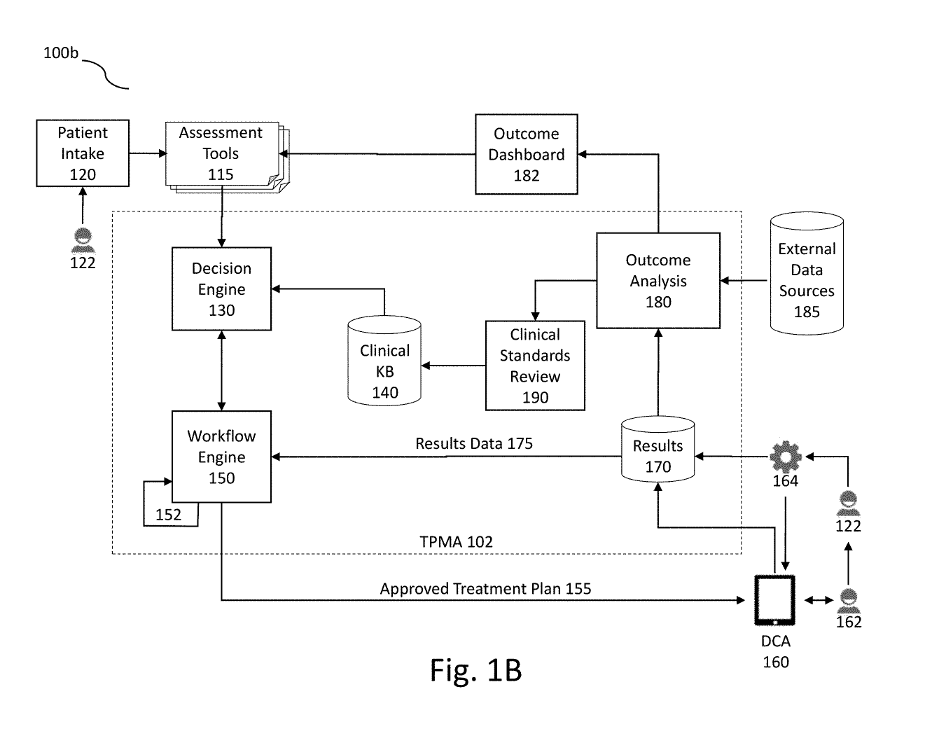

FIGS. 1A-1B illustrate example architectures and systems for implementing a treatment plan management application (TPMA), according to some embodiments of the disclosure. FIG. 1A illustrates a high-level architecture and system 100a for implementing a TPMA 102 (e.g., a clinical intelligence engine). A TPMA 102 is a computerized infrastructure comprising a collection of hardware and software applications in communication with one another to provide a platform for managing treatment plans. A treatment plan may include a set of goals and/or objectives (hereinafter may be referred to as “goals”) that may be used to conduct treatment for a client. Each goal may include a set of target behaviors that a client should master by implementing a series of prescribed exercises/tasks designed to help the client achieve mastery of the target behavior, which therefore improves the client's skill set with respect to the goal associated with the target behavior and exercises.

The TPMA 102 may allow users (e.g., healthcare practitioners) to (a) generate treatment plans based on client assessment information; (b) manage treatment for specific clients such as changing/editing/adjusting treatment goals, manage treatment schedules, view treatment results (e.g., via a dashboard); (c) manage clients, and view a list of client cases assigned to BIs; (d) manage and/or assign BIs to clients; (e) assess the ongoing treatment plan based on results received from implementation of the treatment plans on the client, the results being accessible and available to multiple healthcare professionals in various roles; (f) manage the treatment plan process overall; and/or (g) provide and receive ongoing treatment feedback and/or recommendations.

The TPMA 102 may be deployed in the cloud using cloud-based application servers 116. The TPMA 102 may interface with a Patient Records (system of record) 104 to access client data via web services (e.g., internal networks and/or the Internet). In some embodiments, a medical services provider such as a Kaiser Permanente medical system may provide the patient records 104.

User interfaces, may include, for example, a dashboard 110, a data collection application (DCA) 106, and a parent application 108. The dashboard 110 user interface may allow healthcare practitioners to view a treatment plan and/or results of a treatment plan for a client. The DCA 106 may allow a BI present with a client the ability to review a treatment plan, execute the treatment plan, and input results of the execution of the treatment plan performed on a client. The parent application 108 may provide a parent or guardian of the client with the ability to review a treatment plan for only the client, execute the treatment plan, and input results of the execution of the treatment plan performed on the client. The dashboard 110, DCA 106 and/or parent application 108 may connect to the TPMA 102 via a network, such as, for example, the Internet, WiFi, mobile wireless, etc. The DCA 106 and the parent application 108 may utilize mobile computing devices such as a mobile tablet 114 (e.g., a laptop, a mobile tablet, a mobile phone, etc). In some embodiments, the DCA 106 may include applications such as games and activities for a client to interact with in order to record inputs/results of the client's ability to engage with the activities, thus further providing additional methods of engaging and recording cognitive and behavioral types of responses from a client to a particular treatment plan. The games and activities may correspond to treatment tasks of the overall treatment plan to allow the client to achieve goals defined within the treatment plan.

A provider user interface 112 may provide healthcare providers (e.g., Kaiser Permanente), the ability to view into the client records. The provider user interface 112 may connect to the patient records 104 via the Internet to provide the provider with the ability to view the patient records of the client and the results of the treatment.

FIG. 1B illustrates an architecture and system 100b for implementing a TPMA, according to some embodiments of the disclosure. Those elements equivalent to the embodiment of FIG. 1A are labeled with the same element numbers. System 100b may include a patient intake 120, one or more assessment tools 115, a decision engine 130, a clinical knowledge base 140, a workflow engine 150, DCA 160, internet-of-things sensors 164, a parent application 108 (as shown in FIG. 1A and discussed above), a results database 170, an outcome analysis engine 180, one or more external sources 185, a clinical standards review 190, and an outcome dashboard 182.

A client 122 may be a person diagnosed with, for example, ASD. The client 122 may undergo a patient intake process 120 to determine a current state and severity of ASD. The patient intake process 120 may include accessing an electronic health record system, for example, Kaiser Permanente, to retrieve patient medical history information. The client intake process 120 may include the client being assessed via one or more assessment tools 115, for example, Vineland Adaptive Behavioral Scale, Behavioral Inventory of Executive Function (BRIEF2), Parenting Stress Index (PSI), Verbal Behavior Milestones Assessment and Placement Program (VB-MAPP), etc. Assessment tools 115 may provide assessment information for clinical managers and/or a decision engine 130 to assess the current state of the client. The assessment tools 115 may be integrated with the TPMA 102 system via an application programming interface (API). In some embodiments, assessment tools 115 may be communicatively coupled to the TPMA 102 over a network.

Assessment tools 115 may produce assessment information corresponding to a current state of ASD affecting a client's skill in different categories/domains such as, for example, receptive communications, express communications, pragmatic communication, self-help daily skills, family education, daily living skills, socialization skills, motor skills, and maladaptive behaviors, etc. The assessment information may also include a plurality of scores for the various categories/domains. For example, standard scores for a Vineland-3 assessment may be based on a mean score of 100 with a standard of deviation of 15. Other assessment tools may provide different types of values for scoring a client's skill across different domains.

A decision engine 130 may be an expert system configured to emulate the behavior of the brain function from a connectionism perspective, where neurons are connected to each other via axons, and links are weighted based on relevancy of the problem being solved. For example, Neurons=Nodes=Rules; Axons=Arcs=values. Activations from other neurons are summed at a neuron and passed through an activation function, after which the value is sent to other neurons. The decision engine 130 may generate a candidate treatment plan based on assessment information received from the one or more assessment tools 115. Upon receiving the assessment information comprising the plurality of scores, the decision engine 130 may communicate with a clinical knowledge base 140 to determine goals for the client based at least in part on the plurality of scores, wherein scores below one or more skills threshold targets in any particular domain/category may result in one or more goals being identified for the client 122 to master in order to improve the client's skills within the particular domain/category. In some embodiments, one or more goals may be identified based on scores that are above a normal threshold or below a normal threshold. An example pseudo code of the decision engine 130 may include:

```
FOR each Domain in the Assessment_Report
    IF Assessment_Report.results are below target skill thresholds
    THEN
        Recommend Goals with associated Target Behavior,
          Mastery and Generalization Criteria, and Treatment
          Session directives for a candidate Treatment Plan
    END IF
END FOR
    Forward candidate Treatment Plan to Workflow Engine for approval
END
IF candidate Treatment Plan is modified and returned from
Workflow Engine
THEN
  validate modified Treatment Plan vs Clinical Rules, make changes if
    needed, and forward it back to Workflow Engine until approved
END
```

For example, based on a score below a target skill threshold in the category/domain of “Receptive Communications,” a proposed goal for the client 122 may include a “replacement behavior” skill. A replacement behavior goal is a goal to change a client's negative behavior of, for example, throwing a tantrum when asked to stop playing a video game. Instead of accepting the negative behavior of a child throwing a tantrum, the goal may be to replace the negative behavior with a positive behavior. To achieve the goal, one or more target behaviors may be identified to help the client attain the goal. A target behavior may be an expected response from a client. To help the client 122 achieve a target behavior, the target behavior may include a set of standardized teaching steps/exercises designed to teach the client certain skills to meet the goal. A teaching step may be a specific step that needs to be executed by a client for fulfilling a goal.

Continuing with the replacement behavior goal example, an example target behavior may include exercises such as asking the client to squeeze hands together or asking the client to count to 5 whenever the client is asked to stop playing a video game. Each of the goals may require the client 122 to perform the teaching step/exercise a set number of times, within a period of time, wherein how the client 122 performs the teaching step/exercise is monitored and recorded each time.

The decision engine 130 may generate a candidate treatment plan from the plurality of goals determined from the clinical knowledge base 140. In some embodiments, the decision engine 130 may use an expert system and/or an inference engine. The clinical knowledge base 140 may include a goal bank and a collection of clinical rules associating assessment scores to one or more goals. Each goal may include one or more target behaviors, wherein each target behavior may include one or more teaching skills/exercises/tasks for a client to perform to help the client improve/learn the target behavior. The clinical knowledge base 140 may also information about each assessment tools 115 and how scoring models from each of the assessment tools 115 translate to clinical rules and goals and objectives stored within the clinical knowledge base 140.

An expert system is a computer system that emulates a decision-making ability of a human expert. Expert systems are designed to solve complex problems by reasoning through bodies of knowledge, represented mainly as if—then rules rather than through conventional procedural code. An expert system may be divided into two subsystems: the inference engine and the knowledge base. The knowledge base represents facts and rules (e.g., the clinical knowledge base 140). The inference engine applies the rules to the known facts to deduce new facts. Inference engines may also include explanation and debugging abilities.

The inference engine is an automated reasoning system that evaluates the current state of the knowledge base, applies relevant rules, and then asserts new knowledge from the knowledge base. The inference engine may also include abilities for explanation, so that it may explain to a user the chain of reasoning used to arrive at a particular conclusion by tracing back over the firing of rules that resulted in the assertion.

A clinical rule may include, as an example, if an assessed score for a particular domain for a particular client having a particular client profile is below X, propose goal Y for addressing certain skills that the client lacks or received the low score. In some embodiments, the goals identified may include target behaviors that should be mastered by the client. In some embodiments, the target behaviors may include one or more prescribed exercises/tasks that would help the client attain the target behavior and ultimately, meet the goals identified. In some embodiments, the inference engine may be executed to infer, based on data received from the assessment information and the clinical knowledge base 140, how best to achieve the target behaviors and ultimately, the goals, by determining, for a particular target behavior, whether a forward chaining or a backward chaining of the steps of the exercises/tasks to take with the client would be most beneficial. Current legacy processes include a clinical manager arbitrarily selecting target behaviors to meet certain goals based on the clinical manager's personal experience. The target behaviors and associated teaching steps/exercises may not be the most effective options based on other experiences and/or results from many other clinical managers. Therefore, having an expert engine taking into consideration many results data from a plurality of treatment plans implemented by many other clinical managers that have been verified and validated by a clinical standards review team may provide a more effective treatment plan having target behaviors that are better matched to the intended goals.

The workflow engine 150 may be a data management system configured to manage any workflows. Managing a workflow, in this example of ADS, may include receiving a candidate treatment plan from the decision engine 130, reviewing the candidate treatment plan, providing enhancements to the candidate treatment plan by for example, a program supervisor, validating the enhancements provided by the expert system within the decision engine 130 to ensure the enhancements are within appropriate guidelines, approving the treatment plan by, for example, a program supervisor or a clinical review team, scheduling BIs to execute the one or more goals included in the approved treatment plan, tracking results received from implementation of the treatment plan, and enhancing the treatment plan based on results received from implementation of the treatment plan. In some embodiments, results received from the implementation of the treatment plan (e.g., from the results database 170), may result in an enhancement to the treatment plan, in which case, a recommendation treatment plan may be sent from the workflow engine 150 to the decision engine 130 for evaluation to ensure the recommended treatment plan is within appropriate guidelines. The workflow engine may include a loop 152 of reviewing candidate treatment plans, proposing updates to candidate treatment plans, and validating the updates by the decision engine 130, and eventually approving treatment plans.

The DCA 160 may be software implemented on a mobile computing device such as, for example, a mobile tablet, laptop and/or mobile smart phone that allows a BI 162 present with a client 122 the ability to review a treatment plan, execute the treatment plan, and input results of the execution of the treatment plan on the client. In some embodiments, the DCA 160 may include applications such as games and activities for a client to interact with in order to record inputs/results of the client's ability to engage with the activities, thus further providing additional methods of engaging and recording cognitive and behavioral types of responses from a client to a particular treatment plan.

The DCA 160 may be communicatively coupled with the workflow engine 150 to receive an approved treatment plan 155 for the client 122. The DCA 160 may also be communicatively coupled with a results database 170 for sending results from the implementation/execution of the treatment plan with the client 122. In some embodiments, the DCA 160 may include a display screen of limited size (e.g., a smaller display screen such as a mobile tablet and/or a smart phone). Because of the amount of information available in the treatment of the client 122, the information displayed on the graphical user interface of the DCA 160 is specifically organized in a way that may allow a BI 162 to focus on the goals to work on with the client 122 while providing an all in one view of the teaching steps/exercises/tasks to perform and the ability to input the results of the client 122 performing the exercises/tasks. See below for a more detailed disclosure of a GUI display of the DCA 160. In some embodiments, a similar application to the DCA 160 such as the parent application 108 may be used in conjunction with or in place of the DCA 160 when the parent is providing the treatment with the client and not the BI.

The parent application 108 (as shown in FIG. 1A) may include similar features as the DCA 160, but with a subset of its capabilities. The subset of capabilities may limit the scope of the parent application 108 to display only information about clients under their care. The parent may receive instructions from a clinical manager (CM) via the TPMA 102 to continue care after and/or in-between the BI's treatment. The parent application 108 may allow the client 122 to receive additional treatment interactions with a parent, as opposed to requiring only a BI 162 to be present at the home of the client 122 to implement the treatment plan. The parent application 108 may include similar results recording/capturing features as the DCA 160. The parent application 108 greatly improves the treatment process of ASD because it allows the parents/guardians of the client 122 the ability to participate and help with the execution/implementation of the teaching steps/exercises/tasks to improve the efficacy of the client 122 overall. Furthermore, as the parent inputs results of the client 122 executing the teaching steps/exercises/tasks, the parent inputted results are also stored into the results database 170.

The internet-of-things (TOT) devices 164 may be one or more electronic devices that include one or more sensors for detecting different biometric information of the client 122. For example, a sensor a client 122 may wear on the wrist may provide heartbeat counts per minute, temperature of client 122, etc. in order to detect, as an example, a potential onset of a meltdown of the client 122. As another example, the sensor may be a voice recorder that provides the ability to hear what is going on in the environment of the client 122 during the implementation of the treatment plan at a particular session. The IOT devices 164 may include an AI application that may include rules to interpret biometric information detected and collected by the IOT devices 164 to provide recommendations that may be provided to a BI 162 to be more effective and efficient with the care of the client 122. With the new information being collected by the IOT devices 164, an outcome analysis engine 180, disclosed further below, may generate recommendations of new and/or improved (e.g., updated) clinical rules.

The results data collected by the DCA 160, the IOT devices 164, and the parent application 108 may be sent to and stored in a results database 170. The results database 170 may be located in a cloud infrastructure. In some embodiments, a subset of the results database 170 may be located on the DCA 160, IOT devices 164, and/or the parent application 108 such that results data collected from a treatment session with the client 122 may be uploaded/synchronized with the results database 170 at a later time.

The outcome analysis engine 180 may be an artificial intelligence (AI) engine having machine learning (ML) capabilities to update the clinical knowledge base 140 by updating both its rules and connections over time. The outcome analysis engine 170 may at least recommend new and/or improved (e.g., updated) clinical rules based on information received from the results database 170 and/or industry relevant data from external data sources 185. As treatment plans are implemented by clients on a macro level, the results data accumulating within the results database 170 from a plurality of clients may provide a large amount of meaningful/relevant data points pertaining to treatment plans to allow the AI engine to validate, verify, and confirm an effectiveness ratings of current treatment plans. Furthermore, with an AI engine having ML/deep learning capabilities for analyzing the results data, additional clinical rules, may be developed which may improve the efficacy of the treatments overall.

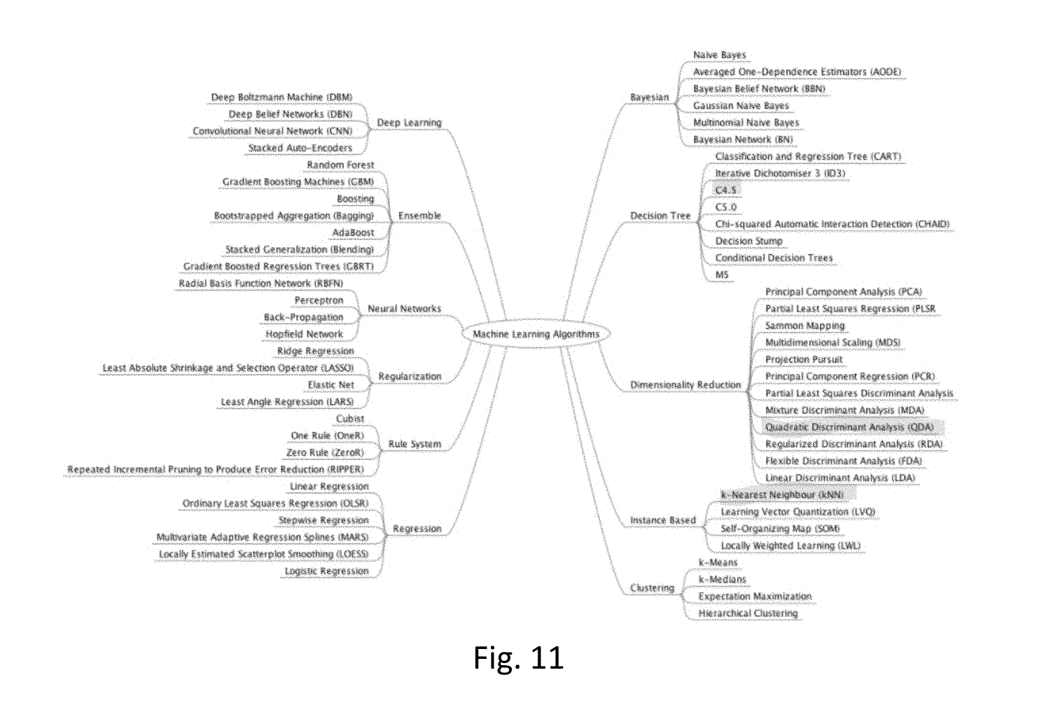

Different ML algorithms may be chosen from a plurality of algorithms based on the types of data available, the volume of data available and the types of problems being solved. Therefore, based on these multi-factors, a particular machine algorithm may be selected to analyze the data to generate recommendations of the new and/or improved (e.g., updated) clinical rules. Examples of machine learning algorithms may include, as examples, C.45, k-NN, QDA, Deep learning, etc. Additional example of ML algorithms may be shown in FIG. 11 and corresponding paragraphs below.

With new results information being collected by the IOT devices 164 (e.g., biometric information), new fine granularity of data points may be collected and correlated with results captured by the DCA 160 and/or parent application 108. For example, during a treatment session, the client 122 may experience a heating up in temperature and an increasing heart rate while performing a particular exercise. With the IOT devices 164 tracking the biometric data of the client 122, along with the results being inputted by the BI 162 and/or the parent via the parent application 108, an AI engine may correlate the data captured by the IOT devices 164 with the results data captured from the DCA 160 or parent application 108. A determination may conclude that implementing a particular exercise, across multiple clients, tends to show an increase in body temperature and an increase in heart rate, which may lead to a possible tantrum. Therefore, this particular exercise should be avoided in a replacement behavior goal that intends to replace a behavior that may lead to a child experiencing a tantrum.

As another example, the new results information collected by the IOT devices 164 may provide additional information about a client's environment that may have influenced an outcome of the exercises based on external triggers created by the environment that may have led to a potential tantrum and/or emotional stress that a BI would normally not recognize during the performance of the treatment.

External data sources 185 may include data sources from patient electronic health records (EHR) and/or industry clinical reviews (e.g., structured data) or from industry specific websites (e.g., unstructured data). The outcome analysis engine 180 may constantly “crawl” external data sources (e.g., the EHR systems, the Internet) for data relevant to at least one of clients, ABA, or ASD treatments. Additionally, or alternatively, the outcome analysis engine 180 may receive industry specific research data sources (e.g., via a subscription) that may provide the outcome analysis engine 180 with structured and/or unstructured data to provide additional data points for correlating results data from the results database 170 with the external data sources to generate recommendations of new and/or improved clinical rules. The recommendations of clinical rules may be reviewed by a clinical standards review team 190, and if approved, be included as new and/or improved rules stored within the clinical knowledge base 140. The outcome analysis engine 180 may receive information from the external data sources 185 and the results data from the IOT devices 164, the DCA 160 and/or the parent application 108, process the received information via a deep learning algorithm to produce the recommended clinical rules.

The recommended clinical rules generated by the outcome analysis engine 180 may be sent to a clinical standards review team 190 for verification, validation and approval to be included in the clinical knowledge base 140. The verification, validation and approval process may include creating a clinical trial for each recommended clinical rule; validating the results of the clinical trials to ensure the recommended clinical rule is effective and operates as recommended by the outcome analysis engine 180; and upon validation that the recommended clinical rules are effective and achieve the desired outcome, the clinical standards review team 190 may approve the recommended clinical rule to be included in the clinical knowledge base 140 for future clients.

The outcome analysis engine 180 may analyze and summarize the results data received for the client 122 to provide an outcome dashboard for a clinical team (e.g., an external case manager) to review and reassess or update candidate treatment plans for the client 122. Treatment plans may be implemented over a period of time (e.g., 6 months, 1 year, multiple years, etc.). Generally, once a treatment plan for a client is approved, the treatment plan is not normally updated or modified during the course of the treatment plan. However, an external case manager via the outcome dashboard 182 may analyze a client's results on a more frequent basis (e.g., monthly, quarterly, semi-annually, annually, etc.) to determine if there is a need to modify or update the treatment plan. Because results data are being captured and analyzed more efficiently with the implementation of the TPMA 102, the external case manager may have access to readily available information to make a determination as to whether or not a current treatment plan should be amended or replaced. If it is determined that a current treatment plan should be replaced, the client 122 may be reassessed using the assessment tools 115 and system 100b may be implemented again.

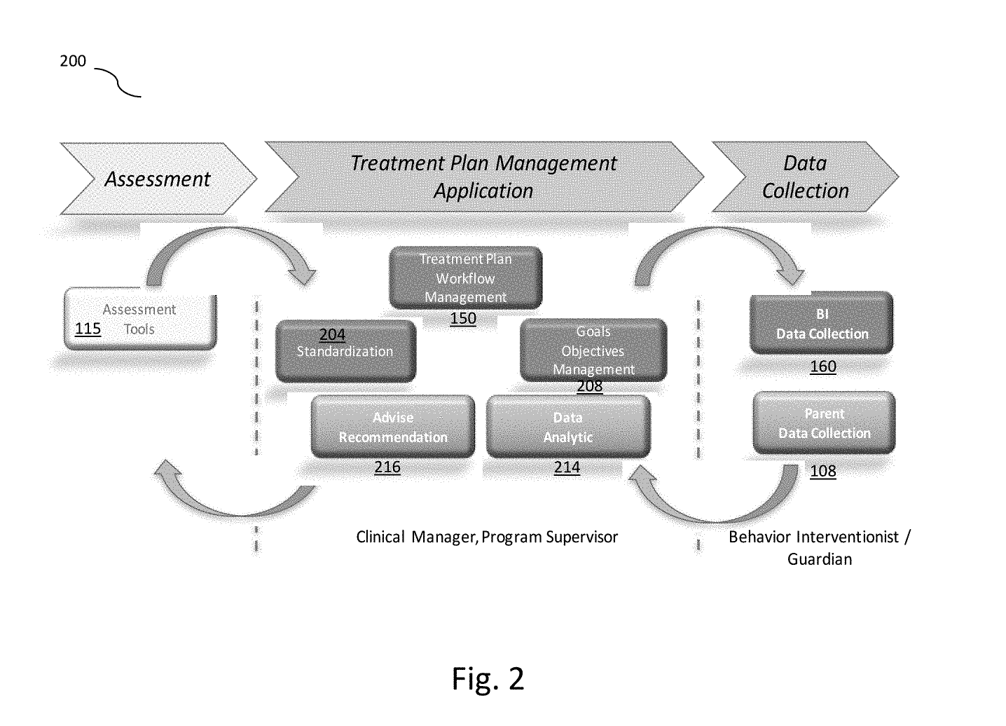

FIG. 2 illustrates a treatment process, according to some embodiments of the disclosure. Those elements equivalent to the embodiment of FIGS. 1A and 1B are labeled with the same element numbers. Process 200 illustrates an improved treatment process including systems and methods to manage and improve the overall treatment process. Assessment tools 115 may be assessment tools disclosed above in FIG. 1B.

Standardization 204 is a standardization of the processes, procedures, elements and objects used in the overall treatment process to ensure users of the system (e.g., practitioners/BIs, clinical managers, program supervisors, clinical review teams, clinical standards review teams, internal and external case managers, funding sources, etc.) are communicating with one another in a standardize format. Information is being created, tracked and analyzed in a standardized way for the purpose of creating a computer systems infrastructure to improve the management and overall efficacy of treatment for clients. Standardization of processes, procedures, elements and objects of any industry may be a first step to automating certain processes and procedures of the industry. The TPMA provides a standardization of the processes and procedures, as well as elements and objects within, as an example, the Applied Behavior Analysis (ABA) industry to help facilitate the management and creation of treatment plans for clients suffering from ASD.

Examples of such standardization may include an entity relationship diagram depicting the relationships of objects such as database table entities in a database to store information in particular way for ABA. Another example may include the decision engine 130 generating candidate treatment plans based on assessment information from assessment tools 115 and clinical knowledge base 140. Yet another example may include the workflow engine 150 assisting in the workflow management of a treatment plan from proposal, to approval, to scheduling the required therapy sessions between BIs and clients, and to managing results data received from implementation of the treatment plans.

Goals and objectives management 208 may be a management of the goals that are to be performed by BI(s) and/or parents/guardians with a client. Each goal may have its own set of conditions. If the conditions are reached for each target behavior, the goal has succeeded. Depending on the goal for skills acquisition or behavioral reduction, the system may automatically present standard structures such as stimulus, response, consequences, antecedence, and behavior. Goals may be prioritized by a clinical manager (CM) or a practitioner supervisor (PS) so that a BI may follow the list goals. However, in some embodiments, the BI may decide/prioritize which goals should be performed at a certain session based on environment or other objectives on at different therapy sessions.

Goals may be grouped into domains. A domain may be a category of goals and may represent a grouping of target behaviors and/or teaching steps/exercises/tasks for reaching a goal. Case managers (e.g., external and internal) and program supervisors may also use the system to search a goal bank for goals by domain. Goals may be generalized, meaning that some of the behaviors may be generalized in a different environment, or with different people. For example, a successful goal achieved with a BI should have similar success with a client's mother or father.

A goal bank may be included within the TPMA solution. The goal bank provides the ability for CMs and Senior CMs to manage goals. For instance, when certain goals are frequently achieved based on a set of target behaviors and teaching steps/exercises/tasks that are associated with the goals, a CM and/or a senior CM may choose to identify such goals as effective goals by indicating a relative success factor to the goals, which may allow the decision engine 130 to select and/or include such goals when generating a candidate treatment plan for clients. In some embodiments, the goal bank may be included within the clinical knowledge base 140.

BI data collection 160 may include the DCA 160 and/or IOT devices 164 discussed above. DCA 160 may be used to make the collection of treatment results process more efficiently by reducing the time BIs spend on administrative tasks such as filling in Session Data Sheets and then inputting this information into Excel worksheets (currently the last 15 minutes of a clients session are spent on clean-up and data recording). Additionally, DCA 160 may serve as a collaboration tool between the different members of a client's treatment team (e.g., CM, PS and BI) that allows the client treatment team to monitor a client's progress against the client's treatment plan and make adjustments if needed on a timely basis. Furthermore, the DCA 160 may assist in defining and designing the platform that will provide an end-to-end solution to track and monitor treatment plans. Parent collection 108 may include the parent application 108 discussed above.

Data analytic 214 may include the outcome analysis engine 180 as discussed above. For example, data analytic may collect data on the activities conducted in each session such as, for example, prompts used by the BI/parent/guardian, time it took for the client to implement a teaching step/exercise/task, a count of the number of times the client implemented the teaching step/exercise/task, number of trials and/or attempts made by the client. The data analytic 214 may automatically calculate session scores and automatically create graphs that show trends over session dates that may include a cumulative record.

Advise recommendation 216 may include the data analytic 214 and/or the outcome analysis engine 180 generating recommendations for new and/or improved (e.g., updated) clinical rules to be pursued by a clinical standards review team 190. The recommendations may be further verified, validated, and approved to become a new and/or improved clinical rule that may be included in the clinical knowledge base 140.

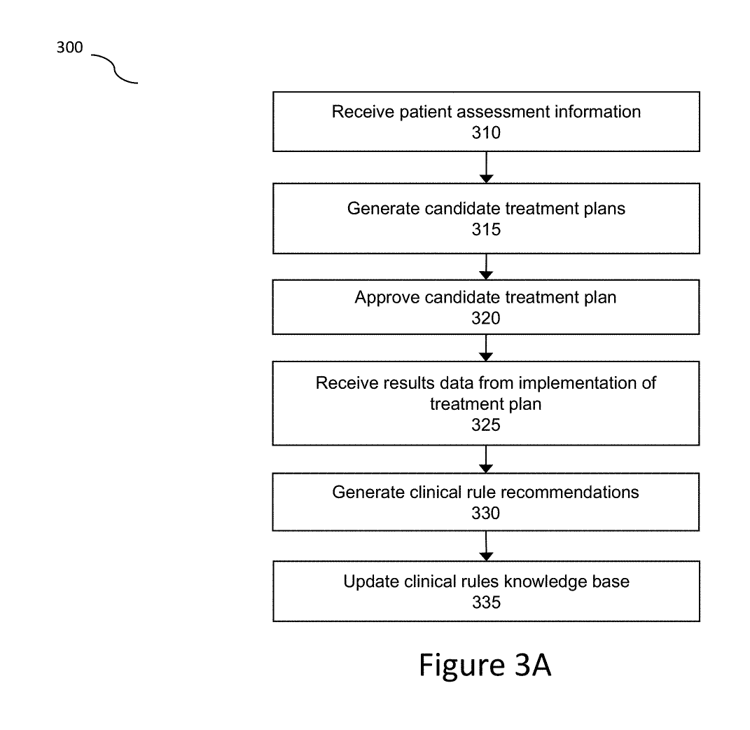

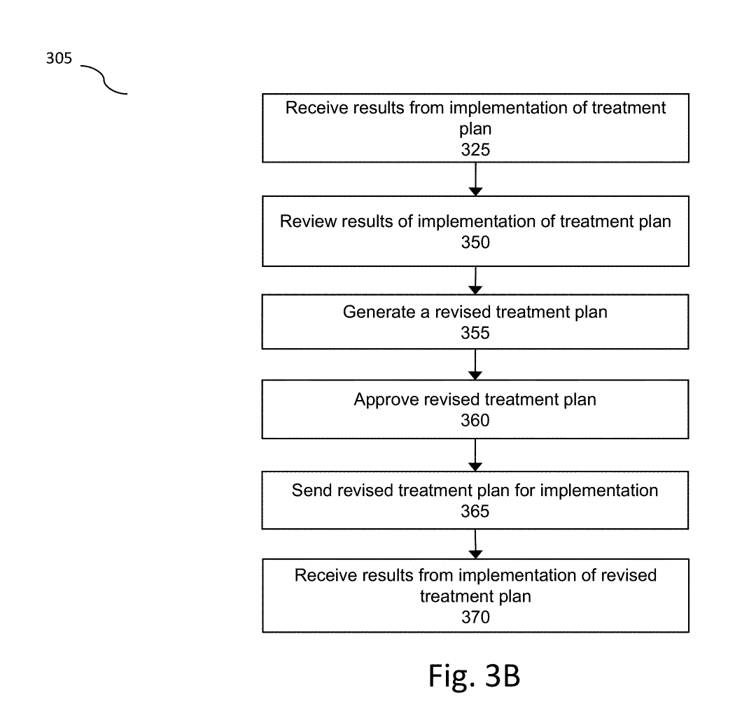

FIGS. 3A-3B illustrate example processes for managing treatment plans, according to some embodiments of the disclosure. FIG. 3A shows a method 300 for managing a treatment plan for a client 122 and generating recommendations of new and/or improved clinical rules based on analysis of at least the results data received from implementation of the treatment plans across a plurality of clients.

At 310, client assessment information may be received by the TPMA 102. In some embodiments, a decision engine 130 may receive the assessment information. The assessment information may include a plurality of scores assessed across a plurality of domains/categories.

At 315, the decision engine may retrieve one or more goals from the clinical knowledge base 140 by executing an expert system having an inference engine to generate a candidate treatment plan based at least in part on the plurality of scores received from the assessment information, as discussed above. The candidate treatment plan may include one or more goals, wherein each goal may include one or more target behaviors, wherein each target behavior may include one or more exercises/tasks for the client to master, the target behavior may also include some mastery criteria to indicate when a goal is met by the client.

At 320, a clinical team assigned to provide care to the client via a workflow engine 150 may approve the treatment plan. Workflow engine 150 may help to provide a single system for the clinical team to interact and collaborate to verify, validate and approve the candidate treatment plan provided by the decision engine 130. In some embodiments, the clinical team may amend or revise the candidate treatment plan as they see fit. However, the revised treatment plan may be sent back to the decision engine 130 for verification and validation to ensure that what was changed and modified by the clinical team is still valid within the parameters of the known facts such as the client's assessed scores, the goals associated to the client's assessed scores, and the target behaviors and teaching steps/exercises/tasks relating to such. If the decision engine 130 determines that the revised treatment plan does not violate any predefined/prescribed rules, then the decision engine 130 may provide an approval check to the clinical team so that the clinical team may ultimately approve the revised and/or the candidate treatment plan. If the decision engine 130 determines that the revised treatment plans violates predefined/prescribed rules, then the decision engine 130 may provide another candidate treatment plan for further approval by the clinical team.

Upon approval of the treatment plan, the workflow engine 150 may assign a BI to the client and schedule the implementation plan of the treatment plan with the BI 162. The scheduling of the implementation may include setting up one or more therapy sessions between the BI with the client over the lifespan of the treatment plan. In some embodiments, the workflow engine 150 may schedule only a portion of the therapy sessions, leaving the other portions of the therapy sessions to be manually scheduled between the BI and the client and/or the parent/guardian of the client. In some embodiments, the workflow engine 150 may assign one or more BIs to a client, depending on the treatment plan and the skills and qualifications of each BI.

At 325, results from the implementation of the treatment plan may be received when the BI via the DCA 160 and/or the parent via the parent application 108 start recording results from the therapy sessions. As the results are captured by the BI and/or the parent(s), the results data may be received and stored within the results database 170. In some embodiments, the results data received may be received real time as the BI and/or the parent(s) capture data to provide the workflow engine real time receipt of the results data 175. This real time receipt of results data may allow a clinical team, located remotely from the therapy session location, the ability to monitor and assess the therapy session.

In some embodiments, the clinical team reviewing the results data may provide recommendations and support to the BI and/or the parent(s) administering the therapy sessions with the client. In some embodiments, the therapy session may simply be a routine, daily session that the parent/guardian administers with the client without the presence of the BI. With the support and monitoring provided by the clinical team located remotely from the location of the therapy session, the parent/guardian may be able to provide a standard of care that is similar to one provided by a BI and thus, reducing the amount of time and therapy sessions a BI may be involved with during the implementation of the treatment plan. This reduction in the need for a BI to be present during a therapy session greatly improves the entire process by allowing BIs to participate in therapy sessions that are more urgent/complicated, while leaving the not so complicated sessions to be performed by the parent/guardian via the parent application 108.

At 330, the results data may be received by the outcome analysis engine 180 for analysis to generate recommendations for new and/or improved (updated) clinical rules to be added to the clinical rules knowledge base. As discussed above, the outcome analysis engine 180 may invoke an artificial intelligence engine to perform analysis (e.g., deep learning) on the results data received from the results database 170. Additionally, the outcome analysis engine 170 may also receive data from external data sources to serve as facts to provide additional input into the AI engine to more accurately analyze the results data to generate recommendations for the new and/or improved clinical rules over time.

At 335, the TMPA 102 may update the clinical rules knowledge base with the recommendations of new and/or improved clinical rules. In some embodiments, prior to updating the clinical rules knowledgebase, the recommendations of the new and/or improved clinical rules may have to be reviewed by a clinical standards review team 190 as discussed above. The clinical standards review team 190 may establish one or more clinical trials for each of the recommendations of new and/or improved clinical rules generated by the outcome analysis engine 180. Once the clinical trials are complete, the results of the clinical trials or any other types of tests may be analyzed by the standards review team 190 for validation and approval of the recommended new and/or improved clinical rules. Upon approval by the clinical standards review team 190, the approved new and/or improved clinical rules may be added and/or updated into the clinical knowledge base 140 for future and current clients.

FIG. 3B shows a method 305 for managing a treatment plan for a client 122 and generating a candidate treatment plan based on results data received during implementation of the treatment plan for the client 122.

At 350, the results data of the implementation of the treatment plan (e.g., from the therapy sessions conducted by either the BI or the parent) may be reviewed by the clinical team assigned to the client 122 via the workflow engine 150. Based on the results data received, the clinical team may suggest that the treatment plan should be modified or revised because, as an example, certain therapy sessions are not producing intended results. At 355, the clinical team may manually generate a revised treatment plan. In some embodiments, the clinical team may use the decision engine 130 to generate a revised treatment plan.

At 360, the revised treatment plan may need to be approved. In order for the revised treatment plan to be approved, the revised treatment plan, via the workflow engine, may be sent to the decision engine 130 for verification and validation. The decision engine 130 may verify that the revised treatment plans are within guidelines specified within the clinical knowledge base 140 for the particular client. The expert system configured within the decision engine 130 may perform the necessary verification and validation of the revised treatment plan to ensure it is within the guidelines for the client 122. Once the decision engine verifies and validates that the revised treatment plan is acceptable, the revised treatment plan may then be approved for the client 122 by the clinical team.

At 365, the workflow engine 150 may send the approved revised treatment plan for implementation by scheduling future therapy sessions with one or more BIs with the client. The one or more BIs having access to the revised treatment plans to conduct the therapy sessions.

At 370, the results data collected from the BI(s) and/or parent(s)/guardian(s) are received into the results database 170 as the treatment plan is being implemented over time. As discussed previously, the results data may be used by the outcome analysis engine to generate new recommendations of clinical rules and/or summary information for the client 122 to be viewed on an outcome dashboard 182 by the clinical team assigned to the client 122.

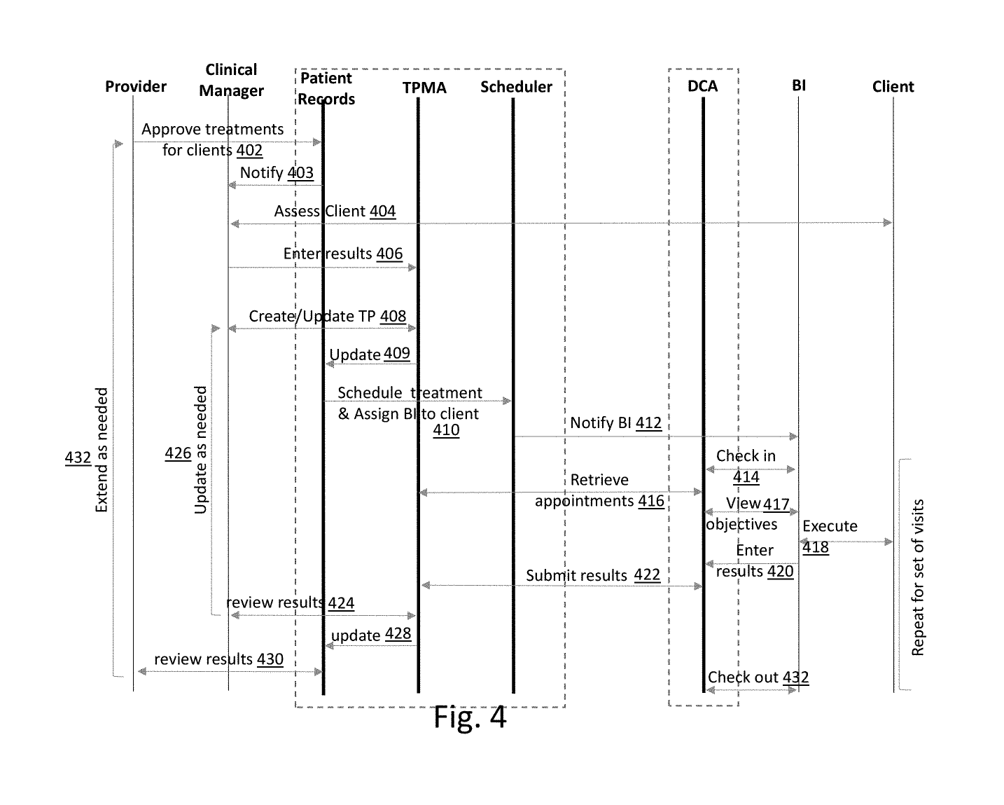

FIG. 4 presents a diagram of an end-to-end interaction process, according to some embodiments of the disclosure. When a provider (e.g., Kaiser Permanente) approves treatments for a client, the provider enters this information into a patient records system at 402. The patient records system may be an external patient records system such as a Kaiser Permanente system that may be external to the cloud-based application servers 116 from FIG. 1A. The clinical manager may receive a notification at 403 and arrange an appointment to perform a client intake process and assess the client at 404. Based on an initial assessment, the clinical manager may determine a treatment strategy and select an assessment tool that may integrate the assessment results with the TMPA at 406.

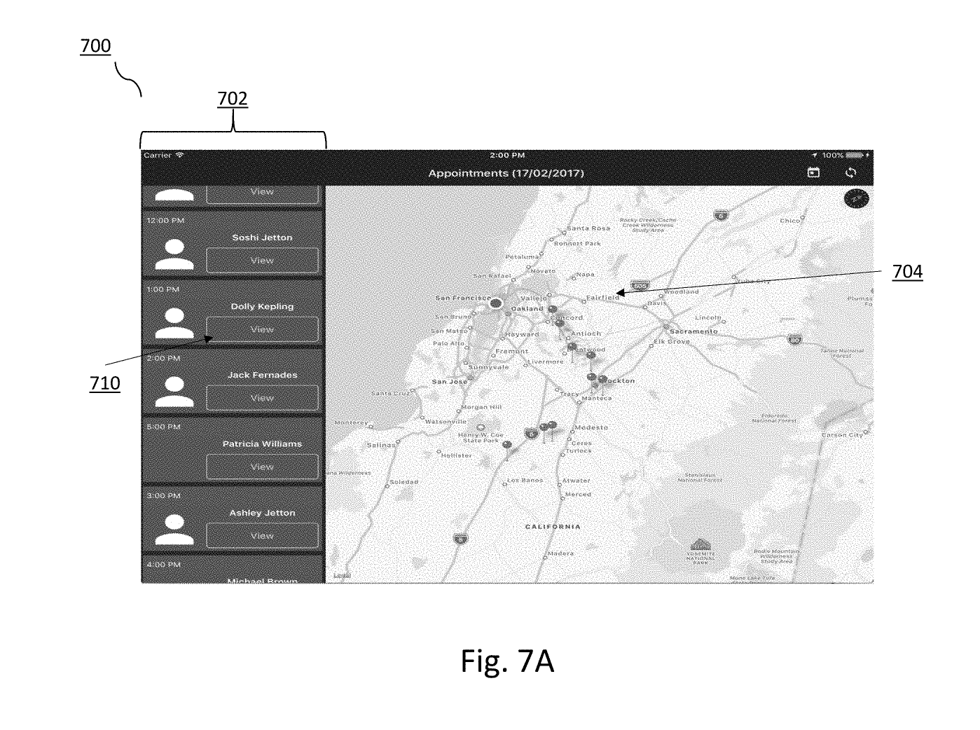

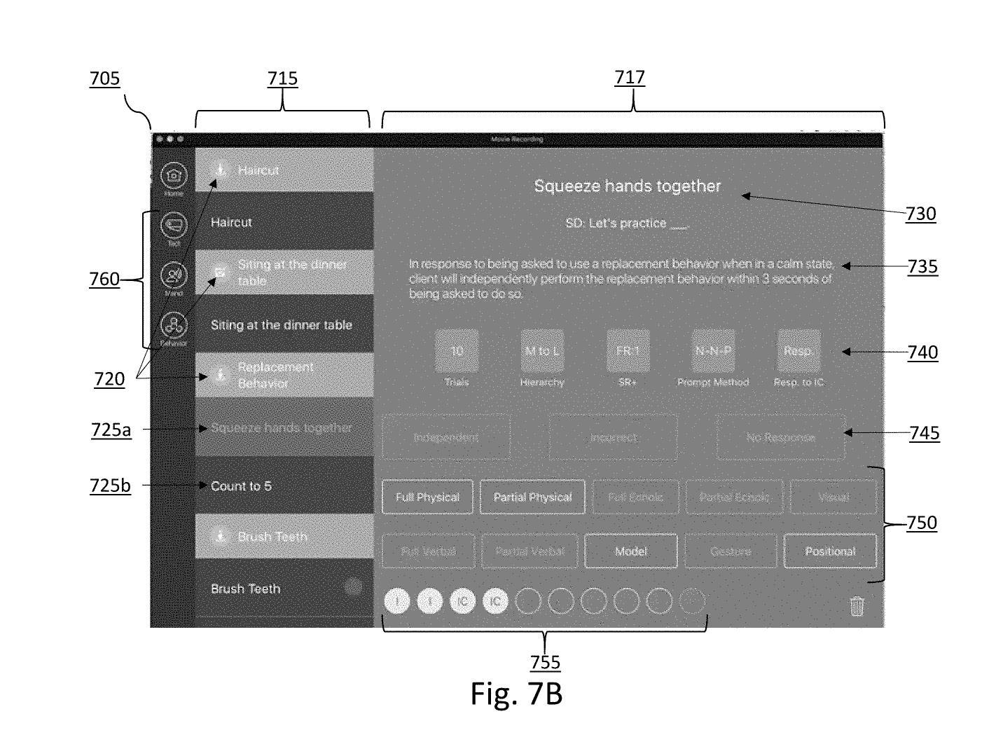

The TPMA may generate a candidate treatment plan at 408. Upon approval of the candidate treatment plan, the patient record system may be updated with the approved treatment plan at 409. A workflow engine may use a scheduler to set up a series of appointments between an assigned BI and the client at 410 and the workflow engine may notify the respective parties at 412 of the appointment. When the BI logs into the DCA at 414, the BI may retrieve the scheduled appointments at 416, which may appear on the DCA screen (e.g., an example of the DCA screen is further illustrated in FIGS. 7A-7B). Associated with each client is a set of goals that make up the approved treatment plan.

The BI may use the DCA to view the treatment plan objectives and execute the treatment plan with the client at 418. For each treatment step/exercise/task, the BI may enter the results into the DCA at 420. When the treatment is completed, the BI may review and submit the results at 422, which are then updated/uploaded to the TPMA. In some embodiments, as the BI is entering the results into the DCA, the results may be immediately updated/uploaded to the TPMA via, as an example, a wireless network or a WiFi network, etc. A clinical manager may review the results at 424 and may decide, based on the results, to re-assess the client, based at least in part on the progress or lack of progress, and thus may adjust the treatment plan as needed at 426. Results may also be sent to and updated in the patient record system at 428 which allows the provider to periodically review results at 430 and possibly approve extensions of the treatment as needed at 432. What has been disclosed in FIG. 4 is an example of an end-to-end process flow, according to an embodiment of the present disclosure. Other embodiments of an end-to-end process flow for a treatment plan management system may exist and that this one embodiment disclosed is just one of many that may be implemented using the disclosed system and methods. One of ordinary skill in the art may appreciate other methods similar to the embodiments disclosed herein.

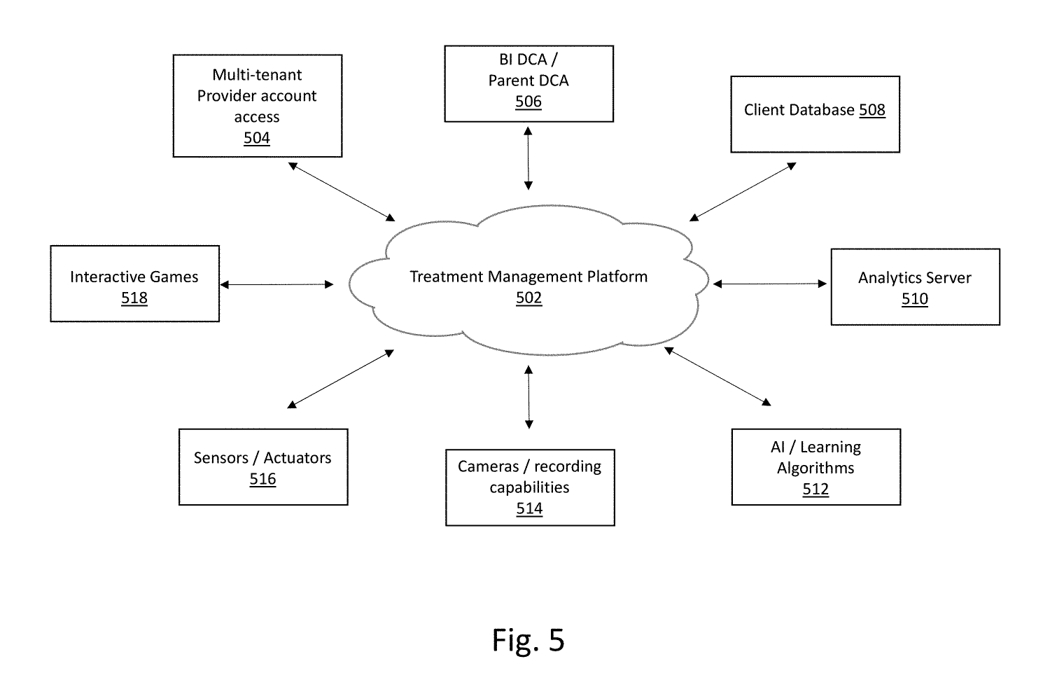

FIG. 5 is a block diagram of an ecosystem of the present disclosure, according to some embodiments. The Treatment Management Platform 502 represents a scalable, cloud-based system that supports the functionality described in this disclosure. The system is designed in a modular way such that most capabilities can be independently supported. Due to the cloud-based architecture, the system may be highly scalable in both the number of clients and stored data. The system also supports a secure multi-tenant architecture, which allows the system to simultaneously support different providers, while maintaining the necessary privacy and regulatory mandates.

The different components of the ecosystem will be discussed. The multi-tenant provider account access 504 represents a strong secure access system that allows multiple tenants to operate within the same system without allowing any of the tenants to access data or functions of another tenant without permission. The DCA/dashboards/Parent DCA 506 provide user interfaces into the system—the DCA represents the UI for the BI(s) to receive assigned treatment plans and to enter treatment result. In addition, the dashboard provides visibility and a UI for treatment plan creation and maintenance by, for example, clinical managers and program supervisors. In some embodiments, other medical professionals may also access the dashboard to provide any type of services related to the treatment plan management.

The parent DCA (e.g., the parent application) provides visibility and a UI for parents to provide input into the system for their client. The types of parent input data may include test results when a parent is administering any portions of the treatment plan on the client. The parent DCA allows a parent or a guardian of the client to review the treatment plans and execute the treatment plans while also providing the ability for the parent or guardian to provide input in the form of results of the execution of the treatment plan upon the client. The client database 508 may be a repository of the clients admitted into the system for treatment. Clients may be associated with different providers.

An analytics server 510 may be used to analyze and visualize treatment data trends, etc. Due to its large scale, the system may provide deep insights into treatment efficacy. Data and results received from multiple clients and multiple treatment plans may be analyzed and provide incredible insight into future treatment plan creations and recommendations. The ability to record and track treatment plan results achieved from the multiple treatment plans may provide medical practitioners insight into which types of treatment plans may produce the greatest results for different types of clients.

The following sets of modules are extensions that provide additional data points and learning capabilities. AI/Learning Algorithms 512 such as, for example deep learning, may be able to improve treatment plan creations by recommending new and/or improved (e.g., updated) clinical rules based on results provided through the large number of treatments performed over time. Sensors and Actuators 516 introduce a family of connected internet-of-things devices to the treatment by providing means to monitor the client and the environment of the client, and based on rules, automatically make adjustment, in order to improve treatment outcomes. The family of connected internet-of-things may also be part of interactive tools and/or toys that may be included as part of treatment programs. The sensors and actuators 516 may take the place of the need to have BIs present at the physical location of a client. Additionally, these sensors and actuators 516 may also provide a stream of input of treatment plan results from the client as the client may be continuously monitored via the sensors and actuators 516 introduced into the client's environment (e.g., a client's home).

Behavioral treatment may greatly benefit from cameras/recording capabilities 514 that enable additional analysis of captured behavioral data like activity, speech, etc., during treatment sessions and/or beyond. This additional type of data can be included in the overall evaluation process, significantly improving the information available to experts and AI learning algorithms 512 for treatment strategies and clinical rules recommendations. Information about the client's physical environment (e.g., room temperature, lighting etc), as well as mentally in the form of behavior of people around the client (e.g., parents or siblings moods, conversation tones etc) can be analyzed as part of the complete treatment program.

Another area of innovation may include interactive games 518 that may provide great insights into behavioral and analytical skills of clients. The interactive games 518 may include games specifically tailored to elicit responses from clients interacting with the games. These specifically tailored games may be developed to correspond to different goals and/or treatment plans. These interactive games 518 may be executed, in some embodiments, on the BI DCA and/or the parent app 506.

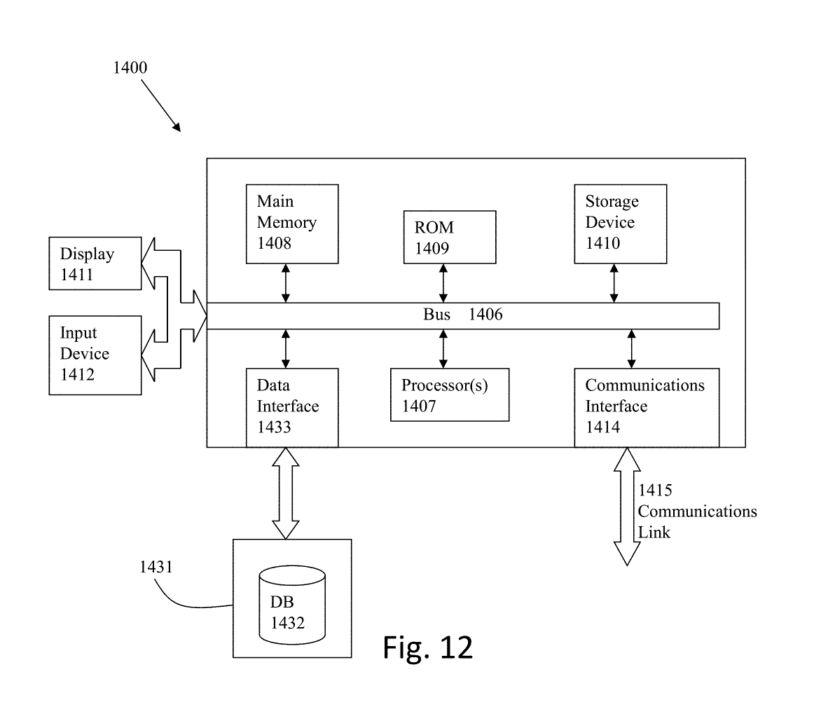

Different actors that may interact with the system are described in Table 1 below. The interactions of some or all of the actors are also illustrated in FIG. 12 below.

TABLE 1

| | SHORT | | |
|---|---|---|---|
| ACTOR | DESCRIPTION | PROVIDES | RECEIVES |
| Senior | Oversees a group | Review/Approve/ | |
| Clinical | of Clinical Manager | Decline approved | |
| Manager | and the day-to-day | Treatment Plan | |
| | operations of a | by Clinical | |
| | clinic which | Manager | |
| | includes Program | | |
| | Supervisor and | | |
| | Practitioner | | |
| Responsible | An adult at least | | |
| Adult | 18 years of age | | |
| | who can sign-off | | |
| | for end of a | | |
| | Treatment | | |
| | Appointment | | |
| | to whom the | | |
| | Client Guardian | | |
| | has given | | |
| | written authority | | |
| | to care for the | | |
| | health and welfare | | |
| | of the Client | | |
| External | Reviews and | | |
| Case | approves the | | |
| Manager | Initial Assessment | | |
| | Report and | | |
| | Progress Report | | |
| | 1222. In all cases | | |
| | they are also the | | |
| | Funding Source. | | |
| Internal | Reviews and | | |
| Case | approves the | | |
| Manager | Initial Assessment | | |
| | Report and | | |
| | Progress Report | | |
| | for submission to | | |
| | Funding Source | | |
| Funding | An medical | | |
| Source | insurance | | |
| | provider who | | |
| | funds for | | |
| | the Treatment | | |
| | for the Client | | |
| Client | A patient who | | |
| 1212 | is under | | |
| | treatment by | | |
| | Practitioner | | |
| Client | A person who | | |
| Guardian | is legally | | |
| | responsible | | |
| | for the client | | |
| Program | Oversees | Creates Treatment | Receives |
| Supervisor | group of | Plan for | Review |
| 1206 | Practitioners | Practitioner. | Request for |
| | | Manages Treatment | a new Goal |
| | | Plan based on | Bank Item |
| | | Clinical | from |
| | | Manager's | Clinical |
| | | comments/advice. | Manager. |
| | | Updates or | |
| | | Approves a | |
| | | Pending Goal | |
| | | Bank Item | |
| | | for Clinical | |
| | | Manager. | |
| Practitioner | Implements | Uses approved | |
| 1210 | Treatment | Treatment Plan | |
| | Plan for | for a Client. | |
| | Client. | Submits | |
| | | results 1224 | |
| | | of each treatment | |
| | | session | |
| | | (Appointment | |
| | | 1218) back | |
| | | to data center. | |
| Clinical | Oversees | Reviews/Approves/ | |
| Manager | group of | Declines | |
| 1204 | Program | candidate | |
| | Supervisors. | Treatment Plan | |
| | | by Program | |
| | | Supervisors | |
| | | Reviews/Approves/ | |
| | | Declines Goals | |
| | | to be added to | |
| | | Goal Bank 1214 | |
| Goal Bank | | | |
| Approver | | | |

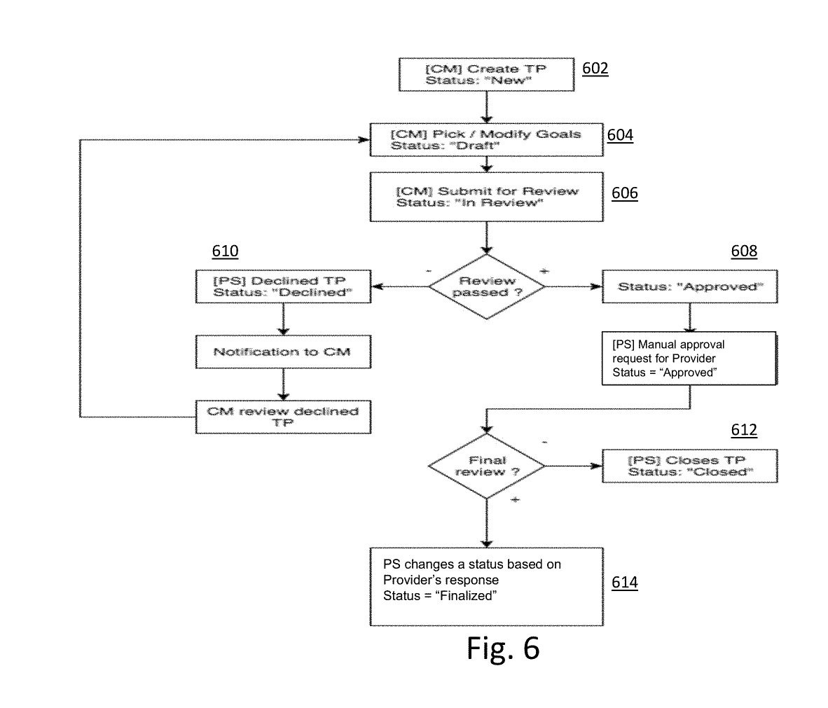

A treatment plan creation process detailing the status of the treatment plan is illustrated in FIG. 6, according to some embodiments. A client may have more than one treatment plan (TP) assigned to them, for example, a main TP and a secondary TP where the secondary TP may be used for a substitute clinical team to work on for approval. At 602, a treatment plan for a client may be created with a status set to “New”. At 604, the status of the TP may be changed to “Draft” as soon as the goals are selected/picked. At 606, the PS may submit the TP for approval to a clinical manager (CM) assigned to the client. The CM may receive a notification that the Draft TP is ready for approval. The CM may provide feedback/suggest changes to the TP. The system may highlight the changes/suggestions made by the CM. If changes are made, the system may notify/send an alert to a PS that feedback from the CM has been received. The PS may make changes and submit the TP again for revision/approval by CM (Status: “In Review”). In some embodiments, the CM may not approve the TP for one reason or another, and the status of the TP may be set to “Declined” at 610. Notifications may be sent to appropriate parties to review the declined and the TP may be sent back to step 604 where goals are selected to possibly provide a TP that may be approved. Once the TP is at an acceptable state, the CM may approve the TP by setting the Status: “Approved” at 608. Once approved, a provider (e.g., a funding source such as, for example, Kaiser Permanente) may conduct a final review of the TP. In some embodiments, at step 612, the provider may not approve the TP, in which case, the status of the TP may be changed to “Closed.” In some embodiments, at step 614, the provider may approve the TP, in which case, the status of the TP may be changed to “Finalized.”

Once created and finalized, a TP may be assigned to a BI, and a series of appointments may be scheduled so that the client and BI may meet periodically to work on a set of defined exercises, as defined in the different goals of the approved/finalized TP. The appointments may take place in a clinical environment or at the client's home.

FIGS. 7A-7B depict example screenshots of a DCA 160, according to some embodiments of the disclosure. FIG. 7A depicts an example screenshot of a DCA appointment screen 700. DCA appointment screen 700 may display an interactive list of client profiles 702 having a scheduled session with the BI for, as an example, Feb. 17, 2017. Additionally, an interactive map 704 may be displayed to the BI to identify locations corresponding to scheduled sessions for the day. In some embodiments, a “push pin” may be dropped at respective locations on the interactive map 704 to identify each scheduled location for the day. The DCA appointment screen 700 allows the BI to quickly glance, and in one display screen, determine for a given day, where each scheduled session will be located and when the BI will need to be at that location. This simple interface may allow the BI to judge the traveling time between each scheduled session. In some embodiments, the interactive map 704 may generate a list to show the travel time between each scheduled session, based at least in part on the distance between the two locations, the time of day, the day of the week, and traffic data. Since the DCA may be executed on a mobile device, design of the GUI interface to display and receive data from the BI takes into consideration, a limited size of screen space available to display and collect data from BI.

The BI may click or touch a “View” button 710 for a particular client from the interactive list of client profiles 702 to drill down into a specific client profile to review a treatment plan prescribed for the respective client.

FIG. 7B depicts an exemplary screen shot of a DCA data collection screen 705 for administering a treatment session, according to some embodiments of the disclosure. For a selected client (e.g., the BI selected a “View” button 710 corresponding to a client from FIG. 7A), a list of goals for treatment of the client may be shown on a left portion 715 of the DCA data collection screen 705. The left portion 715 may display goals 720 and one or more target behaviors 725a-725b associated to a particular goal 720 directly below the respective objective 720. As an example, target behavior 725a may be selected by the BI to begin an exercise with the client to master the target behavior of “Squeeze hands together” to achieve the “Replacement Behavior” goal. Upon the BI selecting target behavior 725a, the details of the exercise to achieve the target behavior 725a may be displayed on the right portion 717 of the DCA data collection screen 705.

The details of the exercise may include a first portion for instructing the BI on how to conduct the exercise, the first portion may include a name of the target behavior 730, instructions 735 on how to administer the exercise, and specific instructions 740 including, for example, a number of times to administer the exercise, an order to administer the steps, a prompt method on how much and when to prompt the client, and how to respond to an incorrect response received from the client. The details of the exercise may include a second portion for allowing the BI to record the client's responses to the exercise, the second portion including buttons 745 for recording the result of the client's response to the exercise.

For example, buttons 745 may include three buttons to be easily selected by the BI to indicate how the client responded to the exercise: (a) independently (e.g., correctly on the client's own without any help from the BI), (b) incorrectly (e.g., incorrectly performing the target behavior), or (c) no response was received from the client after the BI gave the client instructions to elicit the target behavior. The buttons 745 are an example of how a simplified GUI interface may allow the BI to quickly and easily capture the results of the client from the implementation of the teaching step/exercise. This allows the BI to focus on the client and minimize extra administrative confusion in having to manually enter text to record the results of the client. Furthermore, because the DCA may be a mobile computing device with limited screen size, simple buttons that a BI may click on or touch on (e.g., a touchable display screen) makes the data capturing process much more efficient, accurate, and standardized across all BIs.

The results of each trial recorded by the BI choosing/selecting the buttons 745 (e.g., for each execution of the exercise) to record the result of the client's response to the exercise may be displayed as results icon 755, wherein each colored/filled icon indicates a result captured from an execution of an exercise. Furthermore, the results icon 755 may also display the recorded result by displaying I for Independent, IC for Incorrect, or NR for No Response in each of the result icon 755. As an example, the results icons 755 indicate that the “Squeeze hands together” exercise has been conducted four times, with the first two times resulting in an “Independent” result, meaning the client responded correctly and independently for the first two times. However, the next two times, administering the “Squeeze hands together” exercise resulted in two “Incorrect” results. The result icons 755 may also indicate that six more execution of the exercise may still be administered for this target behavior.

Prompting buttons 750 may be buttons that may be activated by state machine running on the DCA to indicate relevant prompting options available to the BI to help the client along, depending on the progress of the session. Prompting buttons 750 are context sensitive. For example, if the client is supposed to squeeze hands together independently, after 3 seconds of no recorded results of an independent result captured by the BI, then prompting buttons “Full Physical”, “Partial Physical”, “Model” and “Positional” may be enabled to allow the BI to capture which prompting type resulted in a response from the client.

Additional details for capturing more specific behavioral actions performed by the client may also be collected using the additional details buttons 760. The additional information may be collected independent of the exercises but may provide additional details on behavioral patterns to be monitored by the BI and/or parent. By capturing the additional information in such a detailed manner, the results data obtained from the DCA and/or the sensors discussed above, may provide the outcome analysis engine 180 (e.g., the AI system) with additional data points to include in the, as an example, multi-variant algorithm to further improve the recommendation of new and/or improved clinical rules.

In some embodiments, if the BI selected target behavior 725b, the display content of the right portion 717 would be for “Count to 5;” a separate target behavior. Because a different target behavior was selected, the right portion 717 may display completely different information with respect to the name of the target behavior 730, instructions on how to administer the exercise 735, specific instructions 740, buttons 745, and additional details buttons 750. Having a graphical user interface display GUI screen designs similar to the DCA data collection screen 705 allows the BI to focus providing treatment and recording treatment for a particular client in a simple one screen user interface. Having a simple GUI design allows the BI the ability to focus on administering the treatments and recording the results with minimal distractions from having to enter text descriptions to describe the results with the DCA since the instructions for executing the treatment exercise and the means for recording the results may all be included on a single page of the GUI without the need to enter/type in text to record the results.

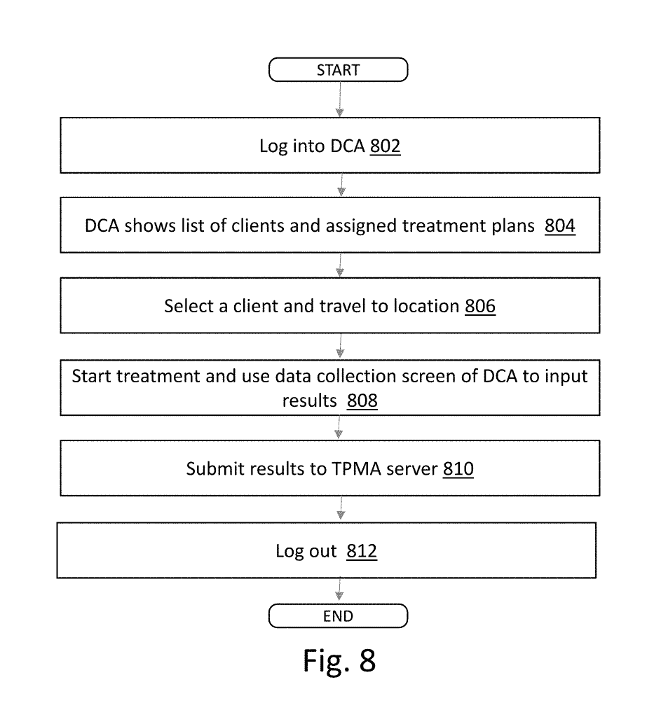

FIG. 8 is a sample flow chart of a BI process of using a DCA, according to some embodiments of the disclosure. At 802, the BI may log into the mobile DCA. At 804, once logged in to the DCA, the BI may be presented with a list of clients and treatment plans associated to each client, wherein each treatment plan may include associated goals. At 806, the DCA may provide location and navigation information, allowing the BI to travel to the location of the client appointment. At 808, the BI may navigate to the data collection screen and start the treatment by following the goals instructions on the screen of the DCA. At 810, depending on the client's response to the exercise, the BI may select buttons displayed on the DCA. Data collected may be stored on the DCA. In some embodiments, the data collected by the DCA may be stored on a persistent storage device in a remote location. Once the treatment has been completed, the BI may review and submit the results to the server. In some embodiments, as the BI records results from the implementation of each exercise, the DCA may immediately store the data collected on the DCA and upload the collected data as results data to the TPMA. The real-time capture and transfer of results data may allow a remotely located clinical team the ability to monitor and support the BI and/or the parent/guardian administering the treatment to the client.

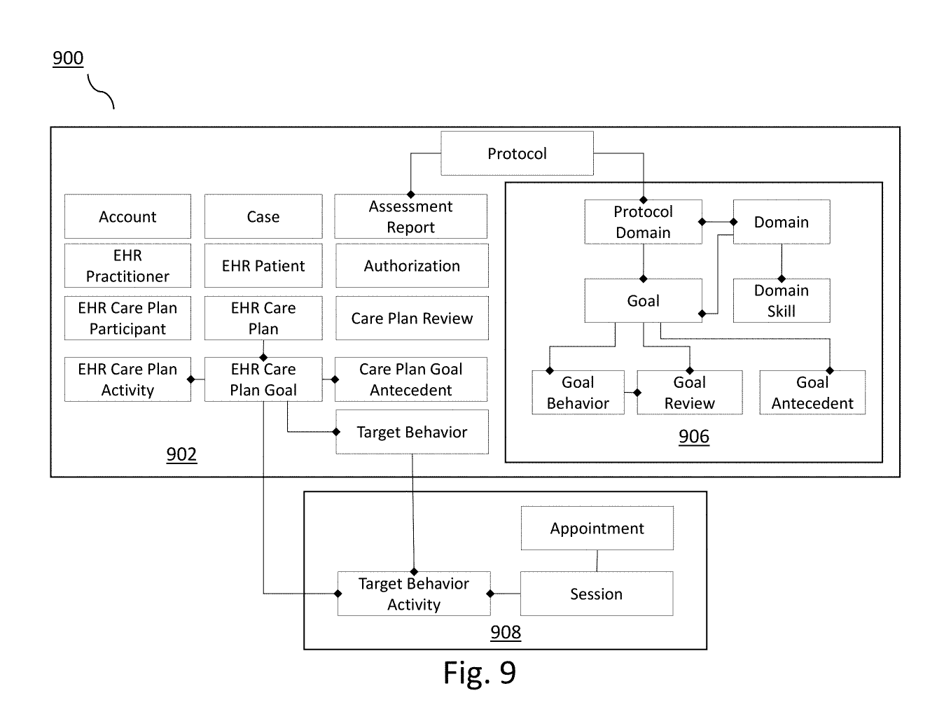

FIG. 9 depicts a block diagram of a Treatment Data Model, according to some embodiments of the disclosure. The treatment data model 900 may be an example of a data model for implementing embodiments of this disclosure. The treatment data model 900 may include TPMA business objects 902, EHR business objects, goal bank business objects 906, and DCA business objects 908. The TPMA business objects 902 may include business objects such as account and case from customer relationship management (CRM) applications or enterprise resource planning (ERP) applications, wherein an account may be a client account and a case may be a treatment plan for the client. The EHR business objects may be common business objects found in systems such as, for example, Health Cloud from SalesForce.com.

TPMA business objects 902 are business objects for managing and implementing the TPMA platform. Common business objects such as Account and Case help to identify the client and the different cases/health related cases, such as for example a treatment plan, associated to the Account (e.g., client). As a part of managing a treatment plan, the TPMA may also include certain business objects to also manage the client 122 within the TPMA. The TPMA business objects may also include Assessment Report, EHR Practitioner, EHR Patient, EHR Care Plan Participant, EHR Care Plan, EHR Care Plan Activity, EHR Care Plan Goal, Authorization, Care Plan Review, Care Plan Goal Antecedent, and Target Behavior. As it may be evident, there are a few EHR specific business objects that allows the TPMA and the EHR systems to integrate seamlessly. By providing similar EHR business objects within the TPMA data model to EHR business objects in external EHR systems, the amount of computer processing for transforming business objects having different data structures for the purpose of integrating the two systems may be greatly reduced when business objects between two systems are common and/or shared. Having common and/or shared business objects between different systems may also help improve the overall speed of processing of data between the two systems as well because there may not be a transforming step to transform business objects from a first data structure to a second data structure in order for the two systems to integrate seamlessly.

Goal bank business objects 906 may comprise the goals for ASD clients to attain. As discussed above, a particular goal may be associated with one or more behavior, wherein each behavior may be associated with one or more teaching steps/exercises/tasks for a client to perform with the intention of having the client achieve a target behavior once the teaching steps/exercises/tasks are mastered. The goal bank business objects may include data structures for storing the goals and/or clinical rules. In some embodiments, the clinical rules may be associated to various assessment score ranges. In some embodiments, the clinical rules may be associated to assessment scores above or below a target threshold. One of ordinary skill in the art may appreciate clinical rules may be associated to assessment scores in many different ways and what is currently disclosed are just a few examples of how different assessment scores may be associated to different clinical rules. The goal bank business objects 906 may also include additional data structures (e.g., goal antecedent) to assist an expert system using an inference engine generate candidate treatment plans.

DCA business objects 908 are business objects that may be persisted in a local storage device of the DCA 160 (from FIG. 1B). DCA business objects 908 persisted on the DCA 160 may include business objects necessary for implementing the DCA functionalities for a BI and/or a parent via a parent application 108. The TPMA and the DCA may be tightly coupled since the DCA may feed results data/session data to the TPMA while the TPMA may send approved/finalized treatment plans and scheduled sessions to the DCA. Because the two systems are so closely integrated, in some embodiments, the DCA business objects 908 may include duplicated TPMA business objects such as account, cases, target behavior, etc. for specific clients that the BI operating the DCA may be associated/assigned with.

Table 2 below depicts a list of Business Objects that may be used in order to organize and store information relevant to the TPMA, according to some embodiments. The list of Business Objects depicted in Table 2 is an example list of Business Objects and is not a full representation of all of the Business Objects of the present disclosure. Each Business Object in Table 2 may include a short description and in some cases, a system of record is identified to further denote which system of record the Business Object may be associated with.

TABLE 2
Business Objects
Business Objects

| | | System |
|---|---|---|
| | | of |
| Object | Short Description | Record |
| Antecedent | Stimulus preceding behavior. | |
| Antecedent | The condition that controls the | TPMA |
| Condition | presence or absence of the behavior. | |
| Assessment | The assessment methodology | TPMA |
| Tool Source | and tool used for creating | |
| | Initial Assessment Report | |
| Behavior | Is generally described as | |
| | “everything one does.” | |
| Behavior | Term used to designate the teaching | |
| Acquisition | and acquiring of skills not | |
| | currently existing in the Client's | |
| | behavioral repertoire | |
| Behavior | The process of ensuring a behavior | |
| Generalization | can and will occur when presented | |
| | with unlearned stimuli. | |
| Behavior | The process of ensuring a behavior | |
| Maintenence | can and will occur when probed | |
| | after a period absent of direct | |
| | teaching of that skill. | |
| Behavior Plan | A document detailing proactive | |
| | and reactive measures for behavior | |
| | targeted for reduction. Will also | |
| | contain replacement behaviors for | |
| | skill acquisition. Produced as a | |
| | result of a Functional Behavior | |
| | Assessment. | |
| Consequence | Stimulus change immediately | |
| | following a response. | |
| Curriculum | Template that generates a | |
| | standardized treatment plan. | |
| Diagnostic | An evaluation completed by | |
| Evaluation | the Funding Source or licensed | |
| | medical provider | |
| Dimensional | Is similar to the First Response | |
| Quality | Trial recordings for prompt levels; | |
| Recording | however, the data recorded is a | |
| | dimensional quantity. | |
| Domain | Category of Objective, | TPMA |
| | grouping Objectives together. | |
| Facesheet | Client Profile/Bio | TPMA |
| First | Is a data collection method of | |
| Response | recording the client's performance | |
| Trial | of the first trial within each | |
| Recording | Instructional Condition targeted | |
| | for treatment. | |
| Fixed Interval | Client is reinforced for the first | |
| | correct response subsequent an | |
| | identified duration of time | |
| Fixed Ratio | Requires an identified number of responses | |
| Reinforcement | prior to the client receiving reinforcement. | |
| Goal | Task to be performed by | TPMA |
| | Practitioner with a Client | |
| Goal Bank | Provides ability for Clinical Manager | TPMA |
| | and Senior Clinical Manager to manage goals. | |
| Goal | Standardized goal in different | TPMA |
| Generalization | environment with different Clients. | |
| Initial | Report produced by the treatment | |
| Assessment | team summarizing direct Assessment | |
| Report | Tool Source results, observation | |
| | findings such as a preference | |
| | and behavioral assessment, and | |
| | initial Treatment Plan | |
| | recommendations. Also contains | |
| | requested Authorization hours. | |
| Instructional | General term for all individuals, | |
| Condition | settings, and stimuli that will be | |
| | programed into treatment | |
| Mand Training | The process of teaching a Client to | |
| | mand for (or demand/request) items, | |
| | actions, or other environmental | |

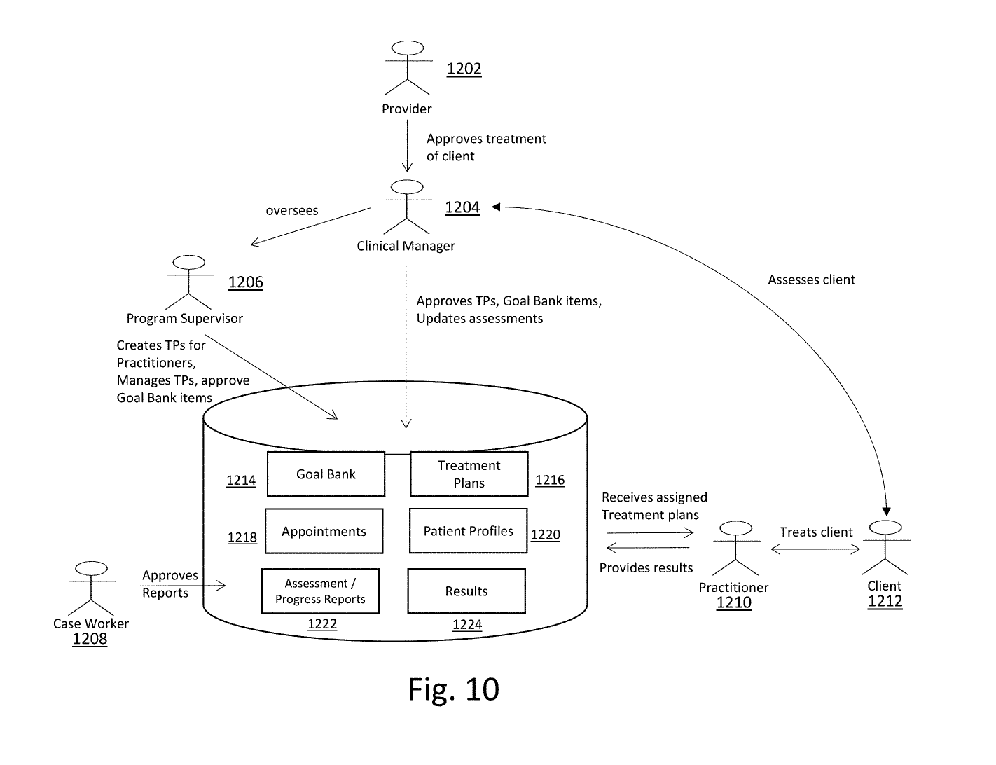

FIG. 10 is a block diagram of a Treatment Process Interactions, according to some embodiments of the disclosure. A provider 1202 (e.g., Kaiser Permanente) may approve treatment for a client 1212. A clinical manager 1204 may approve candidate treatment plans, update assessments, approve goal bank items for adding new goals to the goal bank 1214, and oversee one or more program supervisors 1206. The clinical manager 1204 may add new goals to the goal bank, such that a new goal added to the goal bank 1214 may include new clinical rules added to the clinical rules knowledge base as well. The clinical manager may participate in a clinical standards review team for reviewing, validating, and approving recommended clinical rules generated by an outcome analytics engine.

A program supervisor 1206 may create treatment plans, via a decision engine running in a TPMA, for BI(s) 1210. The program supervisor 1206 may also manage a plurality of treatment plans 1216 assigned to BI(s) 1210 (similar to a clinical manager). Once treatment plans 1216 are assigned to BI(s) 1210, the workflow engine may schedule therapy sessions with respective BI(s) 1210 and client 1212. The program supervisor 1206 may also approve goal bank items to be added to the goal bank 1214.

BI(s) 1210, using a DCA configured to communicate with the TPMA, may retrieve client profiles 1220 and treatment plan 1216 information. Additionally, BI(s) 1210 may use the DCA to implement the treatment plan with the client 1212 while constantly monitoring and providing results data 1224 back to the TPMA by recording results of the therapy session into the DCA during the therapy sessions.

External case manager 1208 may review and approve the initial assessment report and progress reports 1222. In some embodiments, an external case manager 1208 may also be considered a funding source, wherein a funding source may be a medical insurance provider who funds for the treatment for the client.

FIG. 11 depicts an example diagram of different machine learning algorithms suitable for implementing an embodiment of the present disclosure. As discussed above, different ML algorithms utilizing multi-variable processing algorithms may be chosen from a list of algorithms based on the types of data available, the volume of data available and the types of problems being solved. For example, C.45, k-NN, QDA, and/or Deep learning, algorithms may be selected by the outcome analysis engine 180 to analyze the results received from the implementation of the treatment plans and the external data sources such as industry published researches and/or information available on the Internet. Therefore, based on these multi-factors, a particular machine algorithm may be selected to solve a particular problem given the available input data from the results database and external data sources.

Therefore, what has been described is an improved method, system, apparatus and computerized infrastructure for implementing a clinical knowledge base AI expert system to enforce standardization for treatments and outcomes. Additionally, AI machine learning capabilities such as, for example, C4.5, k-NN, QDA algorithms may enhance and expand the clinical knowledge base by using data/information received from feedback from the implementation of the clinical applications and industry published researches. Furthermore, the improved method, system, apparatus and computerized infrastructure also improves the managing of treatment plans over a period of time and creating new clinical rules for generating new treatment plans using a decision engine, a workflow engine, one or more data capture applications, and an outcome analysis engine.

The computerized infrastructure comprising a standardization of processes, procedures, business objects, mobile devices and systems to implement the management and generation of new treatment plans helps to provide continuous care, expandability to include advanced technologies to improve overall treatment. Embodiments of this disclosure enable continuous care through (1) removing spatial and temporal boundaries by utilizing cloud/internet/mobility technologies which allows for home and community-based treatment in addition to the traditional clinical setting treatment; and (2) allowing handoff between healthcare practitioners, which results in enhancement of the quality of care through a continuous management, implementation and monitoring of the treatment plan. Embodiments of the present disclosure discloses a system and methods for managing and improving treatment plans by generating new clinical rule recommendations as results data are received from the implementation of a plurality of treatment plans which allows the efficacy of treatment plans to improve over time.

Various internet-of-things solutions (sensors, actuators) and capturing devices like cameras and microphones may add high value data to the client evaluation and treatment process. For example, the clients' physical environment may be monitored in order to improve treatment efficacy from additional data points not currently captured by the BIs. Having these advanced technologies to capture and record treatment plan results from the client may provide a lot more data capture to reflect treatment plan results in a quick and timely manner, as well as provide a continuous stream of input data for tracking the progress of a client throughout a treatment plan.

Additionally, including emerging Artificial Intelligence and machine learning algorithms that can monitor and improve processes and treatment plans will in the end achieve results in a quicker and more efficiently. Furthermore, including AI at the edge for local decision-making and recommendations may also further improve the speed of processing since the DCA may not be delayed by network latency for sending results data to the TPMA and waiting for recommendations to come back to the DCA. Including AI at the edge device (e.g., DCA and/or parent application) may provide the treatment sessions a faster and more efficient method to process results data without having to deal with network latency issues, especially in locations where WiFi or mobile network connections are not readily available.

What has been disclosed is a non-generic and non-conventional combination of components to solve the technical problem automating a decision making of clinical practitioners to generate new clinical rules based on data that have never been readily available to the clinical practitioners. For example, biometric data collected from the TOT devices provide another dimension of information to be considered in generating new clinical rules in addition to the results data received from the implementations of the treatment plans and the external data sources.

### System Architecture

FIG. 11 is a block diagram of an illustrative computing system 1400 suitable for implementing an embodiment of the present invention. Computer system 1400 includes a bus 1406 or other communication mechanism for communicating information, which interconnects subsystems and devices, such as processor 1407, system memory 1408 (e.g., RAM), static storage device 1409 (e.g., ROM), disk drive 1410 (e.g., magnetic or optical), communication interface 1414 (e.g., modem or Ethernet card), display 1411 (e.g., CRT or LCD), input device 1412 (e.g., keyboard), and cursor control.

According to one embodiment of the invention, computer system 1400 performs specific operations by processor 1407 executing one or more sequences of one or more instructions contained in system memory 1408. Such instructions may be read into system memory 1408 from another computer readable/usable medium, such as static storage device 1409 or disk drive 1410. In alternative embodiments, hard-wired circuitry may be used in place of or in combination with software instructions to implement the invention. Thus, embodiments of the invention are not limited to any specific combination of hardware circuitry and/or software. In one embodiment, the term “logic” shall mean any combination of software or hardware that is used to implement all or part of the invention.

The term “computer readable medium” or “computer usable medium” as used herein refers to any medium that participates in providing instructions to processor 1407 for execution. Such a medium may take many forms, including but not limited to, non-volatile media and volatile media. Non-volatile media includes, for example, optical or magnetic disks, such as disk drive 1410. Volatile media includes dynamic memory, such as system memory 1408.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read.

In an embodiment of the invention, execution of the sequences of instructions to practice the invention is performed by a single computer system 1400. According to other embodiments of the invention, two or more computer systems 1400 coupled by communication link 1415 (e.g., LAN, PTSN, or wireless network) may perform the sequence of instructions required to practice the invention in coordination with one another.

Computer system 1400 may transmit and receive messages, data, and instructions, including program, i.e., application code, through communication link 1415 and communication interface 1414. Received program code may be executed by processor 1407 as it is received, and/or stored in disk drive or other non-volatile storage for later execution.

In the foregoing specification, the invention has been described with reference to specific embodiments thereof. It will, however, be evident that various modifications and changes may be made thereto without departing from the broader spirit and scope of the invention. For example, the above-described process flows are described with reference to a particular ordering of process actions. However, the ordering of many of the described process actions may be changed without affecting the scope or operation of the invention. The specification and drawings are, accordingly, to be regarded in an illustrative rather than restrictive sense.

What is claimed is:

1. A method, comprising:
receiving patient assessment information;
generating a candidate treatment plan by applying the patient assessment information against clinical rules selected from a clinical knowledge base;
approving, by a clinical manager, a treatment plan from the candidate treatment plan via a workflow engine;
receiving results data from implementation of the approved treatment plan with a patient via a mobile computing device;
generating clinical rule recommendations;
approving one or more clinical rules from the generated clinical rule recommendations by a clinical standards review team; and
updating the clinical knowledge base with the one or more clinical rules approved.

2. The method of claim 1, wherein the assessment information comprises a plurality of assessment scores.

3. The method of claim 1, wherein the candidate treatment plan corresponds to clinical rules retrieved from the clinical knowledge base by a decision engine comprising an expert system.

4. The method of claim 3, wherein the clinical knowledge base comprises clinical rules approved and validated by a clinical review team of practitioners reviewing and validating clinical rules generated by an artificial intelligence system analyzing treatment results captured by one or more devices.

5. The method of claim 1, wherein approving the treatment plan comprises analyzing the candidate treatment plan by a clinical manager using a workflow engine, the workflow engine:
verifying the treatment plan based at least in part on the assessment information received; and
assigning and scheduling one or more treatment plan tasks with one or more behavioral interventionists and the patient.

6. The method of claim 1, wherein the results received from the implementation of the treatment plan are received into a results database, the results comprising data received from one or more devices collecting patient information.

7. The method of claim 6, wherein the one or more devices comprise a data collection application (DCA) capturing patient information as a result of the implementation of the treatment plan.

8. The method of claim 6, wherein the one or more devices comprise one or more sensors continuously collecting patient information during the implementation of the treatment plan and/or during a course of normal patient activity.

9. The method of claim 1, wherein the clinical rule recommendations are generated by an outcome analysis engine based at least in part on the results received from implementation of the treatment plan performed on a plurality of patients, the outcome analysis engine comprising a machine learning system utilizing multi-variable processing algorithms for analyzing at least the results received from the implementation of the treatment plan and external data sources comprising structured and unstructured data.

10. The method of claim 9, wherein the multi-variable process algorithm is selected from a plurality of machine learning algorithms based at least in part on (a) data available and (b) a problem a new and/or updated clinical rule intends to solve.

11. The method of claim 1, wherein approving the one or more clinical rules comprises the clinical standards review team validating the clinical rules via one or more clinical trials.

12. A system for managing treatment plans, the system comprising:
a processor;
a memory comprising computer code executed using the processor, in which the computer code implements receiving patient assessment information, generating a candidate treatment plan by applying the patient assessment information against clinical rules selected from a clinical knowledge base, approving, by a clinical manager, a treatment plan from the candidate treatment plan via a workflow engine, receiving results data from implementation of the approved treatment plan with a patient via a mobile computing device, generating clinical rule recommendations, approving one or more clinical rules from the generated clinical rule recommendations by a clinical standards review team, and updating the clinical knowledge base with the one or more clinical rules approved; and
one or more mobile devices capturing results data from implementation of the treatment plan.

13. The system of claim 12, wherein the assessment information comprises a plurality of assessment scores.

14. The system of claim 12, wherein the candidate treatment plan corresponds to clinical rules retrieved from the clinical knowledge base by a decision engine comprising an expert system.

15. The system of claim 14, wherein the clinical knowledge base comprises clinical rules approved and validated by a clinical review team of practitioners reviewing and validating clinical rules generated by an artificial intelligence system analyzing treatment results captured by one or more devices.

16. The system of claim 12, wherein approving the treatment plan comprises analyzing the candidate treatment plan by a clinical manager using a workflow engine, the workflow engine:
verifying the treatment plan based at least in part on the assessment information received; and
assigning and scheduling one or more treatment plan tasks with one or more behavioral interventionists and the patient.

17. The system of claim 12, wherein the results received from the implementation of the treatment plan are received into a results database, the results comprising data received from one or more devices collecting patient information.

18. The system of claim 17, wherein the one or more devices comprise a data collection application (DCA) capturing patient information as a result of the implementation of the treatment plan.

19. The system of claim 17, wherein the one or more devices comprise one or more sensors continuously collecting patient information during the implementation of the treatment plan and/or during a course of normal patient activity.

20. The system of claim 12, wherein the clinical rule recommendations are generated by an outcome analysis engine based at least in part on the results received from implementation of the treatment plan performed on a plurality of patients, the outcome analysis engine comprising a machine learning system utilizing multi-variable processing algorithms for analyzing at least the results received from the implementation of the treatment plan and external data sources comprising structured and unstructured data.

21. The system of claim 20, the multi-variable process algorithm is selected from a plurality of machine learning algorithms based at least in part on (a) data available and (b) a problem a new and/or updated clinical rule intends to solve.

22. The system of claim 12, wherein approving the one or more clinical rules comprises the clinical standards review team validating the clinical rules via one or more clinical trials.

23. A computer program product comprising a non-transitory computer usable medium having executable code to execute a process for managing treatment plans, the process comprising:
receiving patient assessment information;
generating a candidate treatment plan by applying the patient assessment information against clinical rules selected from a clinical knowledge base;
approving, by a clinical manager, a treatment plan from the candidate treatment plan via a workflow engine;
receiving results data from implementation of the approved treatment plan with a patient via a mobile computing device;
generating clinical rule recommendations;
approving one or more clinical rules from the generated clinical rule recommendations by a clinical standards review team; and
updating the clinical knowledge base with the one or more clinical rules approved.

24. The computer program product of claim 23, wherein the assessment information comprises a plurality of assessment scores.

25. The computer program product of claim 23, wherein the candidate treatment plan corresponds to clinical rules retrieved from the clinical knowledge base by a decision engine comprising an expert system.

26. The computer program product of claim 25, wherein the clinical knowledge base comprises clinical rules approved and validated by a clinical review team of practitioners reviewing and validating clinical rules generated by an artificial intelligence system analyzing treatment results captured by one or more devices.

27. The computer program product of claim 23, wherein approving the treatment plan comprises analyzing the candidate treatment plan by a clinical manager using a workflow engine, the workflow engine:
verifying the treatment plan based at least in part on the assessment information received; and
assigning and scheduling one or more treatment plan tasks with one or more behavioral interventionists and the patient.

28. The computer program product of claim 23, wherein the results received from the implementation of the treatment plan are received into a results database, the results comprising data received from one or more devices collecting patient information.

29. The computer program product of claim 28, wherein the one or more devices comprise a data collection application (DCA) capturing patient information as a result of the implementation of the treatment plan.

30. The computer program product of claim 28, wherein the one or more devices comprise one or more sensors continuously collecting patient information during the implementation of the treatment plan and/or during a course of normal patient activity.

31. The computer program product of claim 23, wherein the clinical rule recommendations are generated by an outcome analysis engine based at least in part on the results received from implementation of the treatment plan performed on a plurality of patients, the outcome analysis engine comprising a machine learning system utilizing multi-variable processing algorithms for analyzing at least the results received from the implementation of the treatment plan and external data sources comprising structured and unstructured data.

32. The computer program product of claim 31, the multi-variable process algorithm is selected from a plurality of machine learning algorithms based at least in part on (a) data available and (b) a problem a new and/or updated clinical rule intends to solve.

33. The computer program product of claim 23, wherein approving the one or more clinical rules comprises the clinical standards review team validating the clinical rules via one or more clinical trials.
